(19) United States
(12) Patent Application Publication
(10) Pub. No.: US 2023/0106023 A1
(43) Pub. Date: Apr. 6, 2023

(54) MAMMALIAN CELL CULTURE PROCESSES

(71) Applicant: Boehringer Ingelheim International GmbH, Ingelheim am Rhein (DE)

(72) Inventors: Matthias BRUNNER, Laupheim (DE); Jan BECHMANN, Biberach an der Riss (DE); Elena Joana BOLLGOENN, Erbach (DE); Fabian STIEFEL, Biberach an der Riss (DE); Andreas UNSOELD, Dellmensingen (DE)

(21) Appl. No.: 17/904,487

(22) PCT Filed: Feb. 17, 2021
(86) PCT No.: PCT/EP2021/053859

§ 371 Date: Aug. 18, 2022

(30) Foreign Application Priority Data
Feb. 18, 2020 (EP) 20157829.1

(51) Int. Cl. C12N 5/071; C12N 5/00; C12P 21/00

(52) U.S. Cl. CPC C12N 5/0682; C12N 5/0018; C12P 21/00; C12N 2511/00; C12N 2500/32; C12N 2500/34; C12N 2500/30

(57) ABSTRACT

The present invention relates to the field of cell culture and recombinant protein or recombinant virus production in mammalian cells. It specifically relates to a novel feed medium providing lactate and high concentrations of cysteine and to a method for culturing mammalian cells or for producing a product of interest, such as a heterologous protein or a recombinant virus, using said feed medium.

### FIELD OF THE INVENTION

The present invention relates to the field of cell culture and recombinant protein or recombinant virus production in mammalian cells. It specifically relates to a novel feed medium providing lactate and high concentrations of cysteine and to a method for culturing mammalian cells or for producing a product of interest, such as a heterologous protein or a recombinant virus, using said feed medium.

### BACKGROUND OF THE INVENTION

The majority of recombinant therapeutic proteins in the biopharmaceutical industry are produced by mammalian cell culture due to their capacity for accurate protein folding and post-translational modifications. Within mammalian culture systems, Chinese hamster ovary (CHO) cells are the host of choice in industrial production processes. Their major advantage is their human-like post-translational modification pattern. Furthermore, CHO cells have already proved to be safe hosts and are more likely to be approved for novel therapeutic manufacturing. While the development of stable CHO cell lines with high productivity yielding a high-quality product has been thoroughly done during the past years, there is a constant need for further improvement of cell culture performance. However other mammalian cell lines, such as HEK293, NS0 and BHK21 may also be used in the biopharmaceutical industry for protein expression or virus production.

A major strategy for process development is media design as cells are in constant interaction with their environment. Growth, productivity and product quality are directly influenced by the choice and composition of the used media. The optimization of cell culture medium to fulfill the cells' demand on nutrients and minimize the accumulation of inhibitory substances, has a high impact on process performance.

Not only the media composition has to be observed in detail, deeper understanding of the cells' metabolism is of equally high importance regarding media development. The metabolism of CHO cells and other mammalian cells is characterized by an inefficiently high uptake of substrates such as carbon and nitrogen sources, which leads to high concentrations of cytotoxic or inhibiting byproducts such as lactate, ammonia and various other growth-inhibitory metabolites. Cytotoxic or inhibiting byproducts are particularly problem in fed-batch processes, because the medium is not exchanged and hence cytotoxic byproducts accumulate over time.

With the aim to overcome those negative characteristics of mammalian cells and particularly CHO cells, numerous strategies have been developed and applied, affecting process parameters such as pH, temperature or pCO₂. Moreover, complex feeding strategies and cell line engineering are applied to reduce the formation of inhibiting compounds.

In recent years, bioprocesses with mammalian cells for biopharmaceutical production have been thoroughly developed, focusing mainly on improved growth, productivity and product quality. By usage of a high Seeding Cell Density (SCD), the unproductive growth phase of cells is avoided, leading to an improved space-time yield. However, the demand for optimized nutrient supply of cells by adjusting the media design is even more prominent for cultures with high Seeding Cell Density as such cultures are more prone to accumulating potentially inhibitory or cytotoxic metabolites that may lead to a viability drop, particularly towards the end of the process.

Thus, there is still a growing demand for further development of stable media that support high-density growth of mammalian cell culture and simultaneously support high protein production. Historically, media for cultivation of animal cells included plasma, serum-, or tissue extracts which led to instable and highly inconstant cultivation processes due to the high variability and poor definition of these complex media components and having an inherent risk of viral contamination. Since then, the use of chemically defined serum-free media, which only contain predefined chemical compounds, has increased and are standard in the pharmaceutical industry today.

Cell culture media consist mostly of an energy source such as carbohydrates or amino acids, lipids, vitamins, trace elements, salts, growth factors, polyamines and non-nutritional components such as buffer, surfactants or antifoam agents. Media used in fed-batch cultivations can be divided into two subgroups: Process media (P-media) or basal media and feed media (F-media). Basal media contain all essential components in initial concentration and are used for inoculation. Feed media provide mostly nutrients in high concentrations during the process. Thus, cell culture media are complex compositions of many different compounds and it is a challenge to identify compounds which lead to improved growth, productivity or product quality. One of the main challenges in recent media development is the implementation of media applicable for different cell lines cultivated under different conditions. Media are used under the premise that used cell lines derive from a common host with common expression vector, which implies their similar requirements for nutrient supply. This approach enables a rapid process development by reducing timelines, avoiding cell line specific adjustment of media. Media design has also a great impact on key quality attributes of the desired molecule in upstream manufacturing which further challenges media development, given the wide variety of biopharmaceuticals on the market or in development. Adding to the complexity of media design, different culture techniques imply different demands for the choice of media composition. Optimized feeding strategies or choice of process type result in different demands for supplementation, e.g. in perfusion supporting high viable cell densities (VCD). Thus, cell culture media are complex compositions of many different compounds and it is a challenge to identify compounds which lead to improved growth, productivity or product quality.

Cysteine is a regular component of mammalian cell culture media. It is not considered to be an essential amino acid, but nevertheless an important amino acid for cell culture and protein synthesis. It is known in the art that insufficient cysteine levels lead to a decrease in protein titer. Particularly insufficient levels of Cys in the feed may lead to Cys depletion in the cell. This depletion negatively impacts antioxidant molecules, such as glutathione (GSH) and taurine, leading to oxidative stress with multiple deleterious cellular effects. Although cysteine is known to be an essential component of cell culture media, feeding higher concentration of Cys, however, can lead to improper disulfide bond pairing and increased protein aggregation in the extracellular environment (Ali A. S., et al., Biotechnol. J., 2019, 14: 1800352).

Lactate is on the other hand known as an unwanted by-product which has adverse effects on cell growth and viability. High levels of lactate are reported to have clear negative impacts on cell culture processes, and therefore it was attempted to reduce lactate accumulation and/or to induce lactate consumption in the later stage of cultures (Li J., et al., Biotechnol Bioeng, 2012, 109(5): p 1173-1186). Thus, as described in the prior art, lactate accumulation or lactate production in mammalian cell culture has been avoided (WO 2006/026408, e.g., Example 10, FIG. 42) by media comprising a combination of asparagine and acidic cystine but particularly lactate was not decreased and rather considered to be a waste product and as such not added nor fed to the cell culture. Li et al. used lactate as an alternative feedback pH control strategy and observed for the first time that under lactate consuming metabolic state, feeding exogenous lactate may provide process benefits, particularly reduced ammonium levels and lower CO₂ levels.

Ammonia is a by-product of amino acid metabolism and has a negative impact on cell growth. Other state of the art documents like EP 2135946 A1 and Kishishita et al., J. Biosci Bioeng. (2015), 120 (1), 78-84, disclose cell culture processes with culture media comprising i.a. lactate but explicitly teach that this deems to be an unwanted waste product that should be avoided or kept at low concentrations (see EP 2135946 A1 paragraph [0046] and Kishishita et al., p. 81, left hand colume, 2^{nd} paragraph, lines 1-3).

Ritacco F. V. et al, Biotechnol. Prog., 2018, 34(6): 1407-1426 reviews several approaches of CHO cell culture media development. It discloses in the paragraph bridging pages 1408 and 1410 that lactate as a product of glucose consumption can be inhibitory to cell growth in mammalian cell culture. Respective analyses have shown that in exponential phase, CHO cells largely generate energy via aerobic glycolysis and produce lactate regardless of the concentrations of oxygen, while cells in stationary phase mostly perform oxidative phosphorylation and consume lactate. Yet, it can be derived from this disclosure in general that lactate should be avoided. Further it is said that increased asparagine concentrations could be useful for reducing lactate and ammonium (p. 1412, left hand column, 4th paragraph, lines 18-20), but a role for cystein in this context is not discusssed.

In view of the increasing demand in further improved methods for culturing mammalian cells in fed-batch culture to produce high yields of biopharmaceuticals, including heterologous proteins and recombinant virus, with high product quality there is still a need for improved cell culture media and methods using said cell culture media. The aim of the present invention is therefore to provide an improved fed-batch method for the production of a product of interest in mammalian cells.

### SUMMARY OF THE INVENTION

The present invention relates to the surprising combinational effect of lactate and cysteine on cell culture performance and/or product quality.

In one aspect, a method of producing a product of interest in a fed-batch process is provided comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the product of interest; and (e) optionally isolating the product of interest. Preferably the feed medium is added daily, more preferably continuously. The product of interest is preferably a heterologous protein or a recombinant virus and/or the basal medium and the feed medium is preferably serum-free and chemically defined. In certain preferred embodiment, the molar ratio of lactate/cysteine is about 10:1 to 50:1, preferably about 10:1 to about 30:1.

The lactate may be added at 3 mmol/L/day or higher, at 5 mmol/L/day or higher, at 7 mmol/L/day or higher, or at 10 mmol/L/day or higher. In certain embodiments, the lactate in the cell culture medium is maintained at 0.5 g/L or higher, 1 g/L or higher, 2 g/L or higher, preferably between 2 and 4 g/L.

The cysteine may be provided as cysteine or a salt and/or hydrate thereof, as cystine or a salt thereof or a dipeptide or tripeptide comprising cysteine. Irrespective of the form provided, the cysteine may be added at 0.25 mM/day or higher, at 0.3 mM/day or higher, or at 0.4 mM/day or higher.

In certain embodiments the nucleic acid encodes a heterologous protein and the product titers and/or cell specific productivity is increased compared to the product titers and/or cell specific productivity of the heterologous protein produced by the same method, wherein the feed medium adds cysteine at or below 0.19 mM/day in the absence of lactate. Alternatively or in addition the nucleic acid encodes a heterologous protein and the relative amount of high mannose structures in a population of the heterologous protein is reduced compared to a population of the heterologous protein produced by the same method, wherein the feed medium adds cysteine at or below 0.19 mM/day in the absence of lactate. Preferably the high mannose structures are mannose 5 structures. Alternatively or in addition the nucleic acid encodes a heterologous protein and the relative amount (of total) of acidic species in a population of the heterologous protein is reduced compared to a population of the heterologous protein produced by the same method, wherein the feed medium adds the same concentration of cysteine in the absence of lactate.

The heterologous protein is preferably an antibody or an antigen-binding fragment thereof, a bispecific antibody, a trispecific antibody or a fusion protein. In one embodiment, the antibody, the bispecific antibody or the trispecific antibody is an IgG1, IgG2a, IgG2b, IgG3 or IgG4 antibody.

Also provided is a method of culturing mammalian cells in a fed-batch process comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×day⁻¹) of lactate/cysteine of about 8:1 to about 50:1 to the basal medium resulting in a cell culture medium or to the resulting cell culture medium, wherein the cysteine is added at 0.225 mM/day or higher; and (d) culturing the mammalian cells in the cell culture medium under conditions that allow expression of the product of interest.

In another aspect a method of reducing acidic species in a heterologous protein produced in a fed-batch process is provided comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount of acidic species in a population of the heterologous protein is reduced compared to a population of the heterologous protein produced by the same method wherein the feed medium adds the same concentration of cysteine in the absence of lactate.

In yet another aspect a method of reducing high mannose structures in a heterologous protein produced in a fed-batch process is provided comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount of the high mannose structures in a population of the heterologous protein is reduced compared to a population of the heterologous protein produced by the same method wherein the feed medium adds the cysteine at or below 0.19 mM/day in the absence of lactate, preferably wherein the high mannose structures are mannose 5 structures.

In yet another aspect a method of preventing negative effects of cysteine on product quality characteristics when producing a heterologous protein in a fed-batch process is provided comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein from the mammalian cells; wherein the negative effects on product quality characteristics in a population of the heterologous protein are reduced compared to a population of the heterologous protein produced by the same method wherein the feed medium adds the same concentration of cysteine in the absence of lactate.

The mammalian cell used in the methods according to the invention may be any mammalian cell or cell line, preferably the mammalian cell is a HEK293 cell or a CHO cell or a HEK293 cell or a CHO cell derived cell, preferably the mammalian cell is a CHO cell or a CHO derived cell.

Also provided is a heterologous protein produced by any of the methods according to the invention, preferably by the method of reducing acidic species in a heterologous protein or of reducing high mannose structures in a heterologous protein produced in a fed-batch process as described herein. The heterologous protein may also be produced by the method of preventing negative effects of cysteine on product quality characteristics when producing the heterologous protein as described herein.

In yet another aspect the invention relates to a use of lactate in a feed medium for reducing acidic species in a heterologous protein produced in a fed-batch process, wherein the feed medium adds cysteine at 0.225 mM/day or higher.

Also provided is a use of lactate in a feed medium for reducing high mannose structures in a heterologous protein produced in a fed-batch process, wherein the feed medium comprises cysteine at 0.225 mM/day or higher. Preferably the high mannose structures are mannose 5 structures.

Also provided is a use of lactate in a feed medium for preventing negative effects of cysteine on product quality characteristics of a heterologous protein produced in a fed-batch process, preferably wherein the negative effects on product quality characteristics are increased high mannose structures, increased low molecular weight species and/or increased acidic species.

Also provided is a use of lactate and cysteine in a feed medium for increasing heterologous protein titer and/or cell-specific productivity in a fed-batch process. Preferably the fed-batch process comprises culturing a mammalian cell, wherein the mammalian cell is preferably a HEK293 cell or a CHO cell or a HEK293 cell or CHO cell derived cell, preferably the mammalian cell is a CHO cell or a CHO derived cell.

In yet another aspect a feed medium for mammalian cell fed-batch culture is provided comprising lactate and cysteine at a molar ratio (mM/mM) of lactate/cysteine of about 8:1 to about 50:1. Preferably the feed medium comprises one or more feed supplements for separate addition.

In yet another aspect a kit is provided comprising (a) a concentrated feed medium for mammalian cell fed-batch culture comprising lactate and optionally cysteine, and (b) an aqueous supplement separate from the concentrated feed medium comprising cysteine, wherein the feed medium and the supplement provide a lactate/cysteine molar ratio (mM/mM) of about 8:1 to about 50:1 and cysteine at 0.225 mM/day or higher in a daily addition of less than 5%, preferably less than 3.5% of the cell culture starting volume.

### SHORT DESCRIPTION OF FIGURES

FIG. 1: Viable cell density (A), viability (B), relative IgG titer (C) and lactate concentration (D) of the ultra High Seeding Density (uHSD) processes using a seed concentration of 10×10E06 cells/ml in a 3 L bioreactor. CHO cells were cultures for 13-14 days using a regular uHSD in the presence or absence of bolus addition of lactate and/or cysteine. (C) The IgG concentration on the y-axis is provided as a titer relative to the highest measured value (100%).

FIG. 2: Viable cell densities, viability, product titer and lactate concentration out of the DoE experiment for two cell lines in a regular process in a 250 ml bioreactor. (A, B, C and D) cell cultures of cell line A (CHO-K1; IgG1) were cultivated with 0 g/L/day sodium lactate and 0 ml/l/day cystine (0 Lac/0 Cystine; control), 30 g/L/day sodium lactate and 0.84 ml/L/day of a second cystine feed at 17.2 g/L (30 Lac/0.84 Cystine) and 15 g/L/day sodium lactate and 1.67 ml/L/day of a second cystine feed at 17.2 g/L (15 Lac/1.67 Cystine). (A) Viable cell density [10E06 cells/ml], (B) viability [%], (C) IgG titer relative to the highest measured value [%] and (D) lactate concentration [g/L] is provided. (E, F and G) cell cultures of cell line B (CHO-K1; IgG4) were cultivated with 0 g/L/day sodium lactate and 1.67 ml/L/day of a second cystine feed at 17.2 g/L (0 Lac/1.67 Cystine), 30 g/L/day sodium lactate and 1.67 ml/L/day of a second cystine feed at 17.2 g/L (30 Lac/1.67 Cystine), 30 g/L/day sodium lactate and 0 ml/L/day of a second cystine feed at 17.2 g/L (30 Lac/0 Cystine), and 15 g/L/day sodium lactate and 0.84 ml/L/day of a second cystine feed at 17.2 g/L (15 Lac/0.84 Cystine). (E) Viable cell density [10E06 cells/ml], (F) viability [%], (G) IgG titer relative to the highest measured value [%] and (H) lactate concentration [g/L] is provided.

FIG. 3: Harvest viability for cell line A as a function of lactate and cystine feeding (R²: 0.95; Q²: 0.85). The unit g/L on the y-axis refers to the addition of sodium lactate; the unit ml/L/d on the x-axis refers to the addition of 17.2 g/L cystine.

FIG. 4: Product titer for cell line A as a function of lactate and cystine feeding (R²: 0.98; Q²: 0.96). The unit g/L on the x-axis refers to the addition of sodium lactate; the unit ml/L/d on the y-axis refers to the addition of 17.2 g/L cystine. Highest product titers could be obtained at high lactate and high cystine feeding. The values of titer normalized (%) are normalized to the highest value of the DoE (across the cell lines used in the DoE).

FIG. 5: Acidic peak variants (APG) for cell line A as a function of lactate and cystine feeding (R²: 0.98; Q²: 0.97). The unit g/L on the y-axis refers to the addition of sodium lactate; the unit ml/L/d on the x-axis refers to the addition of 17.2 g/L cystine. The increase in APGs due to cystine feeding can be strongly reduced through additional lactate feeding.

FIG. 6: Harvest viability for cell line B as a function of lactate and cystine feeding (R²: 0.95; Q²: 0.85). The unit g/L on the y-axis refers to the addition of sodium lactate; the unit ml/L/d on the x-axis refers to the addition of 17.2 g/L cystine.

FIG. 7: Product titer for cell line B as a function of lactate and cystine feeding (R²: 0.98; Q²: 0.96). The unit g/L on the x-axis refers to the addition of sodium lactate; the unit ml/L/d on the y-axis refers to the addition of 17.2 g/L cystine. Highest product titers could be obtained at high lactate and high cystine feeding. The values of titer normalized (%) are normalized to the highest value of the DoE (across the cell lines used in the DoE).

FIG. 8: Acidic peak variants (APG) for cell line B as a function of lactate and cystine feeding (R²: 0.98; Q²: 0.97). The unit g/L on the y-axis refers to the addition of sodium lactate; the unit ml/L/d on the x-axis refers to the addition of 17.2 g/L cystine. The increase in APGs due to cystine feeding can be strongly reduced through additional lactate feeding.

FIG. 9: Mannose 5 structures (Man5) for cell line A (A) and cell line B (B) as a function of lactate (R²: 0.93; Q²: 0.77). The unit g/L on the x-axis refers to the addition of sodium lactate. Confidence intervals (95%) are presented as dotted lines.

FIG. 10: Low Molecular Weight Species (LMWs) for two cell lines as a function of cystine and lactate. (A and B) Low Molecular Weight Species (LMWs) for cell line A as a function of (A) cystine, indicated as ml/L/d on the x-axis referring to the addition of 17.2 g/L cystine, and (B) lactate, indicated as g/L on the x-axis referring to the addition of sodium lactate. (C and D) Low Molecular Weight Species (LMWs) for cell line B as a function of (C) cystine, indicated as ml/L/d on the x-axis referring to the addition of 17.2 g/L cystine, and (D) lactate, indicated as g/L on the x-axis referring to the addition of sodium lactate. Confidence intervals (95%) are presented as dotted lines. The values LMWs norm. (%) on the y-axis are normalized to the highest value of the DoE.

FIG. 11: Viable cell density (A), viability (B), lactate concentration (C) and relative IgG titer (D) for cell line C are shown. 14.37 g/L cystine in an extra feed at 2 ml/L/day (w Cys) or 30 g/L lactate together with the free feed medium at 30 ml/L/day (w Lac) or both (w Cys/Lac) were added to the cell culture. Control cells were only fed with the feed media (Feed 1).

FIG. 12: Viable cell density (A), viability (B), lactate concentration (C) and relative IgG titer (D) for cell line D are shown following treatment as described in the Figure legend of FIG. 11.

FIG. 13: Viable cell density (A), viability (B), lactate concentration (C) and relative IgG titer (D) for cell line E are shown following treatment as described in the Figure legend of FIG. 11.

FIG. 14: Viable cell density (A), viability (B), lactate concentration (C) and relative IgG titer (D) for cell line F are shown following treatment as described in the Figure legend of FIG. 11.

FIG. 15: Product titer for cell line A as a function of lactate and cystine feeding (R2: 0.84; Q2: 0.77) in DoE optimization of uHSD processes. Highest product titers could be obtained at high lactate and high cysteine feeding. The values of titer normalized (%) are normalized to the highest value of the DoE (across the cell lines used in the DoE).

FIG. 16: Product titer for cell line B as a function of lactate and cystine feeding (R2: 0.84; Q2: 0.77) in DoE optimization of uHSD processes. Highest product titers could be obtained at high lactate and high cysteine feeding. The values of titer normalized (%) are normalized to the highest value of the DoE (across the cell lines used in the DoE).

FIG. 17: Harvest viability for cell line A as a function of lactate feeding (R2: 0.94; Q2: 0.89). Confidence intervals (95%) are presented as dotted lines.

FIG. 18: Harvest viability for cell line B as a function of lactate feeding (R2: 0.94; Q2: 0.89). Confidence intervals (95%) are presented as dotted lines.

FIG. 19: Acidic peak variants (APG) for cell line A as a function of lactate and cystine feeding (R2: 0.99; Q2: 0.97). The increase in APGs due to cysteine feeding can be strongly reduced through additional lactate feeding.

FIG. 20: Acidic peak variants (APG) for cell line B as a function of lactate and cystine feeding (R2: 0.99; Q2: 0.97). The increase in APGs due to cysteine feeding can be strongly reduced through additional lactate feeding.

FIG. 21: Mannose 5 structures (Man5) for cell line A as a function of lactate (R2: 0.71; Q2: 0.48). Confidence intervals (95%) are presented as dotted lines.

FIG. 22: Mannose 5 structures (Man5) for cell line B as a function of lactate (R2: 0.71; Q2: 0.48). Confidence intervals (95%) are presented as dotted lines.

### DETAILED DESCRIPTION OF THE INVENTION

The general embodiments “comprising” or “comprised” encompass the more specific embodiment “consisting of”. Furthermore, singular and plural forms are not used in a limiting way. As used herein, the singular forms “a”, “an” and “the” designate both the singular and the plural, unless expressly stated to designate the singular only.

The term “cell cultivation” or “cell culture” includes cell cultivation and fermentation processes in all scales (e.g. from micro titer plates to large-scale industrial bioreactors, i.e. from sub mL-scale to >10.000 L scale), in all different process modes (e.g. batch, fed-batch, perfusion, continuous cultivation), in all process control modes (e.g. non-controlled, fully automated and controlled systems with control of e.g. pH, temperature, oxygen content), in all kind of fermentation systems (e.g. single-use systems, stainless steel systems, glass ware systems). According to the invention the cell culture is a mammalian cell culture and is a fed-batch culture. In a preferred embodiment the cell culture is a cell culture in a volume of >1 L, preferably >2 L, >10 L, >1.000 L, >5000L and more preferably >10.000 L.

The term “fed-batch” as used herein relates to a cell culture in which the cells are fed continuously or periodically with a feed medium containing nutrients. The feeding may start shortly after starting the cell culture on day 0 or more typically one, two or three days after starting the culture. Feeding may follow a preset schedule, such as every day, every two days, every three days etc. Alternatively, the culture may be monitored for cell growth, nutrients or toxic by-products and feeding may be adjusted accordingly. In general, the following parameters are often determined on a daily basis and cover the viable cell concentration, product concentration (titer) and several metabolites such as glucose, pH, lactate, osmolarity (a measure for salt content), and ammonium (growth inhibitor that negatively affects the growth rate and reduces viable biomass). Compared to batch cultures (cultures without feeding), higher product titers can be achieved in the fed-batch mode. Typically, a fed-batch culture is stopped at some point and the cells and/or the medium is harvested and the product of interest, such as a heterologous protein or a recombinant virus is isolated and/or purified. A fed-batch process is typically maintained about 2-3 weeks, e.g., about 10-24 days, about 12 to 21 days, about 12 to 18 days, preferably about 12 to 16 days. Particularly, a fed-batch process for the production of a heterologous protein is typically maintained about 2-3 weeks, e.g., about 10-24 days, about 12 to 21 days, about 12 to 18 days, preferably about 12 to 16 days.

For recombinant virus production, cells are typically transduced with the recombinant virus at the desired cell density. Feeding may start shortly after starting the cell culture on day 0 or more typically one, two or three days after starting the culture, wherein the cells are transduced with the recombinant virus at the desired cell density at cell inoculation or after a certain period of time when the desired cell density is achieved, which may be after the feeding has started, such as at days 1-7 after starting the culture, preferably at days 2-5 after starting the culture, more preferably at days 3-5 after starting the culture. Alternatively, the cells may be stably transfected with one or more nucleic acid molecule encoding the recombinant virus or the cell may be transiently transfected with one or more nucleic acid molecule encoding the recombinant virus or a combination thereof. Like for virus transduction of the mammalian cells, for transient transfection the cells may be transfected with one or more nucleic acid encoding the recombinant virus at the desired cell density at cell inoculation or after a certain period of time when the desired cell density is achieved, which may be after the feeding has started, such as at days 1-7 after starting the culture, preferably at days 2-5 after starting the culture, more preferably at days 3-5 after starting the culture.

By definition any nucleic acid, sequences or genes introduced into a host cell are called “heterologous nucleic acid” “heterologous sequences”, “heterologous genes”, “heterologous RNAs” or “transgenes” or “recombinant gene” with respect to the host cell, even if the introduced sequence is identical to an endogenous nucleic acid, sequence or gene in the host cell. A “heterologous” or “recombinant” protein or RNA is thus a protein or RNA expressed from a heterologous nucleic acid, sequence or gene, preferably a DNA. In a preferred embodiment, the introduced nucleic acid, sequence or gene is not identical to an endogenous nucleic acid sequence or gene of the host cell in question.

The term “encodes” and “codes for” as used herein refers broadly to any process whereby the information in a polymeric macromolecule is used to direct the production of a second molecule that is different from the first. The second molecule may have a chemical structure that is different from the chemical nature of the first molecule. For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

The terms “polypeptide” or “protein” are used interchangeably. These terms refer to polymers of amino acids of any length. These terms also include proteins that are post-translationally modified through reactions that include, but are not limited to glycosylation, glycation, acetylation, phosphorylation, oxidation, amidation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with similar or modified properties. Amino acid modifications can be prepared for example by performing site-specific mutagenesis or polymerase chain reaction mediated mutagenesis on its underlying nucleic acid sequence. The terms “polypeptide” and “protein” thus also include, for example, fusion proteins consisting of an immunoglobulin component (e.g. the Fc component) and a growth factor (e.g. an interleukin), antibodies or any antibody derived molecule formats or antibody fragments.

The term “product of interest” as used herein refers to any product produced in a mammalian cell, particularly to a heterologous protein and a recombinant virus.

The term “cell culture medium” as used herein is a medium to culture mammalian cells comprising a minimum of essential nutrients and components such as vitamins, trace elements, salts, bulk salts, amino acids, lipids, carbohydrates in a preferably buffered medium. Typically a cell culture medium for mammalian cells has an about neutral pH, such as a pH of about 6.5 to about 7.5, preferably about 6.8 to about 7.3, more preferably about 7. Non limiting examples for such cell culture media include commercially available media like Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma) etc. as well as proprietary media from various sources. The cell culture medium may be a basal cell culture medium. The cell culture medium may also be a basal cell culture medium to which the feed medium and/or additives have been added. The cell culture medium may also be referred to as fermentation broth, if the cells are cultured in a fermenter or bioreactor.

The term “basal medium” or “basal cell culture medium” as used herein is a cell culture medium to culture mammalian cells as defined below. It refers to the medium in which the cells are cultured from the start of a cell culture run and is typically not used as an additive to another medium, although various components may be added to the basal medium. The basal medium serves as the base to which optionally further additives (or supplements) and/or a feed medium may be added during cultivation, i.e., a cell culture run resulting in a cell culture medium. The basal cell culture medium is provided from the beginning of a cell cultivation process. In general, the basal cell culture medium provides nutrients such as carbon sources, amino acids, vitamins, bulk salts (e.g. sodium chloride or potassium chloride), various trace elements (e.g. manganese sulfate), pH buffer, lipids and glucose. Major bulk salts are usually provided only in the basal medium and should not exceed a final osmolarity in the cell culture of about 280-350 mOsmo/kg, so that the cell culture is able to grow and proliferate at a reasonable osmotic stress.

The term “feed” or “feed medium” as used herein relates to a concentrate of nutrients/ a concentrated nutrient composition used as a feed in a culture of mammalian cells. Thus, it is provided as a concentrate that is added into the cell culture. It is provided as a “concentrated feed medium” to minimize dilution of the cell culture, typically a feed medium is provided at 10-50 ml/L/day, preferably at 15-45 ml/L/day, more preferably at 20-40 ml/L/day and even more preferably at 30 ml/L/day based on the culture starting volume (CSV, meaning the start volume on day 0) in the vessel. This corresponds to a daily addition of about 1-5%, preferably about 1.5-4.5%, more preferably about 2-4% and even more preferably about 3% of the culture starting volume. For cultures using high density seeding or ultra-high density seeding higher feeding rates may be beneficial such as 10-50 ml/L/day, 15-45 ml/L/day or 25-45 ml/L/day. This corresponds to a daily addition of about 1-5%, about 1.5-4.5%, or about 2.5-4.5% of the culture starting volume. The feeding rate is to be understood as an average feeding rate over the feeding period. A feed medium typically has higher concentrations of most, but not all, components of the basal cell culture medium. Generally, the feed medium substitutes nutrients that are consumed during cell culture, such as amino acids and carbohydrates, while salts and buffers are of less importance and are commonly provided with the basal medium. The feed medium is typically added to the (basal) cell culture medium/fermentation broth in fed-batch mode. The feed medium added (repeatedly or continuously) to the basal medium results in the cell culture medium. The feed may be added in different modes like continuous or bolus addition or via perfusion related techniques (chemostat or hybrid-perfused system). Preferably, the feed medium is added daily, but may also be added more frequently, such as twice daily or less frequently, such as every second day. More preferably the feed medium is added continuously. The addition of nutrients is commonly performed during cultivation (i.e., after day 0). In contrast to the basal medium, the feed medium typically consists of a highly concentrated nutrient solution (e.g. >6×) that provides all the components similar to the basal medium except for ‘high-osmolarity-active compounds’ such as major bulk salts (e.g., NaCl, KCI, NaHCO₃, MgSO₄, Ca(NO₃)₂). Typically a 6×-fold concentrate or higher of the basal medium without or with reduced bulk salts maintains good solubility of compounds and sufficiently low osmolarity (e.g. 270-1500 mOsmo/kg, preferably 310-800 mOsmo/kg) in order to maintain osmolarity in the cell culture at about 270-550 mOsmo/kg, preferably at about 280-450 mOsmo/kg, more preferably at about 280-350 mOsmo/kg. The feed medium may be added as one complete feed medium or may comprise one or more feed supplements for separate addition to the cell culture. The use of one or more feed supplements may be necessary due to different feeding schedules, such as regular feeding and feeding on demand as often performed for glucose addition, which is therefore typically at least also provided as a separate feed. The use of one or more feed supplements may also be necessary due to low solubility of certain compounds, solubility at different pH of certain compounds and/or interactions of compounds in the feed medium at high concentrations. The feed medium is preferably chemically defined (optionally comprising a recombinant protein, such as insulin or IGF). It does not contain cells, has not been in contact with cells in culture or does not contain cell derived metabolic waste products. Thus, as used herein, the term “feed medium” excludes a pre-conditioned medium derived from a cell culture or a culture medium in cell culture, i.e., in the presence of cells (also referred to as cell culture medium herein).

The term “feed supplement” as used herein relates to a concentrate of a nutrient, which might be added to the feed medium before use or may be added separately from the feed medium to the basal medium and/or the cell culture medium. Thus, a compound may be provided with the feed medium or the feed supplement or a compound may be provided with the feed medium and the feed supplement. For example, cysteine may be added in a two-feed strategy with the feed medium and the feed supplement. As the feed medium, the “feed supplement” is provided as a concentrate in order to avoid dilution of the cell culture.

The cell culture medium, both basal medium and feed medium is preferably serum-free and chemically defined. The basal medium and/or the feed medium may further be protein-free. A “serum-free medium” as used herein refers to a cell culture medium for in vitro cell culture, which does not contain serum from animal origin. This is preferred as serum may contain contaminants from said animal, such as viruses, and because serum is ill-defined and varies from batch to batch. The basal medium and the feed medium according to the invention are serum-free.

A “chemically defined medium” as used herein refers to a cell culture medium suitable for in vitro cell culture, in which all components are known. More specifically it does not comprise any supplements such as animal serum or plant, yeast or animal hydrolysates. A chemically defined medium is therefore also serum-free. The basal medium and the feed medium according to the invention are preferably chemically defined. In one embodiment the basal medium and/or the feed medium are serum-free and chemically-defined and optionally comprises a recombinant growth factor such as insulin or insulin-like growth factor (IGF). The basal medium and/or the feed medium as referred to herein comprise no further proteins, except for, once in cell culture to provide the cell culture medium, proteins produced by the mammalian cell to be cultured.

A “protein-free medium” as used herein refers to a cell culture medium for in vitro cell culture comprising no proteins (except for proteins produced by the cell to be cultured once in cell culture), wherein protein refers to polypeptides of any length, but excludes single amino acids, dipeptides or tripeptides. Specifically, growth factors such as insulin and insulin-like growth factor (IGF) are not present in the medium. Preferably, the basal medium and feed medium according to the present invention are chemically defined and protein-free.

The term “viability” as used herein refers to the % viable cells in a cell culture as determined by methods known in the art, e.g., trypan blue exclusion with a Cedex device based on an automated-microscopic cell count (Innovatis AG, Bielefeld). However, there exist of number of other methods for the determination of the viability such as fluorometric (such as based on propidium iodide), calorimetric or enzymatic methods that are used to reflect the energy metabolism of a living cell e.g. methods that use LDH lactate-dehydrogenase or certain tetrazolium salts such as alamar blue, MTT (3-(4,5-dimethylthiazol-2-yl-2,5-diphenyltetrazolium bromide) or TTC (tetrazolium chloride).

The term “producing” or “highly producing”, “production”, “production and/or secretion”, “producing”, “production cell” or “producing at high yield” as used herein relates to the production of a product of interest, such as a heterologous protein or a recombinant virus, encoded by a nucleic acid. An “increased production and/or secretion” or “production at high yield” relates to the expression of the product of interest, such as the heterologous protein or the recombinant virus, and means in the context of the heterologous protein an increase in cell specific productivity, increased titer, increased overall productivity of the cell culture or a combination thereof. In the context of a recombinant virus it means an increase in cell specific and/or total produced particles, an increase in cell specific and/or total infective particles or a combination thereof. Increased titer as used herein relates to an increased concentration in the same volume, i.e., an increase in total yield and may be used for a heterologous protein as well as a recombinant virus.

The term “enhancement”, “enhanced”, “enhanced”, “increase” or “increased”, as used herein, generally means an increase by at least about 10% as compared to control cell culture, for example an increase by at least about 20%, or at least about 30%, or at least about 40%, or at least about 50%, or at least about 75%, or at least about 80%, or at least about 90%, or at least about 100%, or at least about 200%, or at least about 300%, or any integer decrease between 10-300% as compared to a control cell culture. As used herein, a “control cell culture” or “control mammalian cell culture” is a cell culture using the same cell (same cell clone) producing the same product using the same method according to the invention, wherein the feed medium adds cysteine at or below 0.19 mM/day in the absence of lactate.

### Methods of Producing a Product of Interest

In one aspect the invention relates to a method of producing a product of interest in a fed-batch process comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the product of interest; and (e) optionally isolating the product of interest. Preferably the product of interest is a heterologous protein or a recombinant virus, more preferably a heterologous protein.

Also provided is a method of culturing mammalian cells in a fed-batch process comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; and (d) culturing the mammalian cells in the cell culture medium under conditions that allow expression of the product of interest. Optionally the product of interest may further be purified or isolated. Preferably, the product of interest is a heterologous protein or a recombinant virus, more preferably a heterologous protein.

The feed medium used in the methods according to the invention is added daily, preferably continuously during the feeding period of the fed-batch process. In one embodiment the feed medium is added starting from days 0 to 5. The person skilled in the art will understand that this may also depend on the seed density. For a normal seeding density (0.7 to 1×10⁶ cells/ml) feeding is typically started at days 1-5, preferably days 2-3. Feeding is typically continued until at least 5 days before the end of the fed-batch process, until at least 4 days before the end of the fed-batch process, until at least 3 days before the end of the fed-batch process, until at least 2 days before the end of the fed-batch process and preferably until the end of the process. More preferably feeding is started at days 2-3 and is continued at least until 2 days before the end of the fed-batch process, more preferably until the end of the cell fed-batch process. For high seeding densities (>1 to 4×10⁶ cells/ml) feeding is typically started at days 0-4, preferably days 0-2. Feeding is typically continued until at least 5 days before the end of the fed-batch process, until at least 4 days before the end of the fed-batch process, until at least 3 days before the end of the fed-batch process and may be continued until the end of the process. Preferably feeding is started at days 0-2 and is continued until at least 4 days or 3 days before the end of the fed-batch process. For ultrahigh seeding densities (>4 to 20×10⁶ cells/ml) feeding is typically started at days 0-3, preferably days 0-1. Feeding is typically continued until at least 5 days before the end of the fed-batch process, until at least 4 days before the end of the fed-batch process, until at least 3 days before the end of the fed-batch process and may be continued until the end of the process. Preferably feeding is started at day 0 and is continued until at least 4 days or 3 days before the end of the fed-batch process. Thus, depending on the start of the feed medium addition the method may further comprise a step bi) between steps b) (inoculating the mammalian cells) and c) (adding a feed medium), wherein step bi) comprises culturing the mammalian cells in the basal medium. The unit “mmol×L⁻¹×day⁻¹” as used herein for defining the addition of lactate or cysteine may also be referred to as mmol/L/day or mM/day. It refers to the mmol/L provided per day, irrespective of whether the addition is a bolus addition or a continuous addition. According to the invention the cells are preferably in a lactate consuming metabolic state in step (c) and/or when lactate is added to the culture.

The term “inoculating” as used herein refers to collecting a sample of mammalian cells, such as of a mammalian cell line, and placing them into a medium that contains the nutrients needed for growth. Typically, the mammalian cells are placed into a basal medium for growth or production. This step may also be referred to as seeding. The mammalian cells may be inoculated into the basal medium at different seeding densities. As referred to herein the terms “seeding” or “normal seeding” refer to a standard seeding density of about 0.7 1×10⁶ cells/ml to about 1×10⁶ cells/ml, the term “high seeding” refers to a seeding density of greater 1×10⁶ cells/ml to about 4×10⁶ cells/ml and the term “ultrahigh seeding” refers to a seeding density of greater 4×10⁶ cells/ml to about 20×10⁶ cells/ml or even higher, preferably of about 6×10⁶ cells/ml to about 15×10⁶ cells/ml, more preferably of 8×10⁶ cells/ml to about 12×10⁶ cells/ml.

The molar ratio of lactate/cysteine may be about 10:1 to 50:1, preferably about 10:1 to about 30:1, preferably about 15:1 to about 30:1.

In one embodiment, the lactate is added at 3 mmol/L/day or higher, at 3.8 mmol/L/day or higher, at 5 mmol/L/day or higher, preferably at 7 mmol/L/day or higher, at 7.8 mmol/L/day or higher, at 10 mmol/L/day or higher or even at 15 mmol/L/day or higher.

The lactate in the cell culture medium is maintained at 0.5 g/L or higher, 1 g/L or higher, preferably at 2 g/L or higher, preferably between 2 and 4 g/L, more preferably between 2 and 3 g/L. The concentration in the cell culture medium should be maintained below 5 g/L (˜56 mM), where lactate becomes toxic. Thus, the lactate concentration in the cell culture medium is maintained between about 1 g/L and about 4.5 g/L (˜10 to 50 mM), between about 2 g/L and about 4 g/L (˜20-45 mM), and preferably between about 2 g/L and about 3 g/L (˜20-35 mM). The lactate (MW=89.07 g/mol) may be provided as a salt, an ester and/or a hydrate thereof and/or as lactic acid, preferably a salt, such as sodium lactate (MW=112.06 g/mol), wherein 1 g/L of lactate equates to about 1.25 g/L of sodium lactate. Exemplary esters of lactate are e.g., ethyl lactate or butyl lactate. Lactic acid may also be used in the context of the present invention. However, it may affect the pH of the feed medium and hence it is preferably titrated with NaOH to provide sodium lactate prior to addition to or mixing with the components of the feed medium. The salt, ester and/or hydrate of lactate or the lactic acid is provided at an equimolar concentration to the lactate concentration provided herein. In a preferred embodiment the basal medium does not contain lactate added as a medium component. However, lactate may be generated during culture in the basal medium as a metabolite. The lactate has been obtained as sodium lactate or as lactic acid, which has been titrated with NaOH to provide sodium lactate prior to addition to the feed medium. The term “lactate” as used herein refers to L-lactate. Thus, e.g., sodium lactate and lactic acid refer to sodium L-lactate and L-lactic acid.

The cysteine may be provided as cysteine or a salt and/or a hydrate thereof, as cystine or a salt thereof or as a dipeptide or tripeptide comprising cysteine. The cysteine salt and/or hydrate or the cystine or a salt thereof or the dipeptide or tripeptide comprising cysteine is provided at an equimolar concentration to the cysteine concentrations provided herein. The terms “cysteine” and “cystine” as used herein refer to L-cysteine and L-cystine. According to the invention the cysteine is added at 0.225 mM/day or higher, wherein the cysteine is added to the basal medium resulting in the cell culture medium or to the resulting cell culture medium. Preferably the cysteine is added at 0.25 mM/day or higher, at 0.3 mM/day or higher, more preferably at 0.4 mM/day or higher, more preferably at 0.5 mM/day or higher. In one embodiment the cysteine is added from about 0.225 mM/day to about 0.6 mM/day, from about 0.25 mM/day to about 0.6 mM/day, from about 0.3 mM/day to about 0.6 mM/day, or from about 0.4 mM/day to about 0.6 mM/day.

Without being bound by theory, cysteine is used in protein synthesis and for glutathione (GSH) production. Glutathione acts as an important cellular antioxidant, maintaining cellular redox balance, by removal of reactive oxygen species (ROS). ROS are chemically highly reactive and a byproduct of oxygen metabolism. During oxidative stress, ROS levels rise and enhance damage of RNA and proteins as well as promoting apoptosis. Hence, adding cysteine could maintain the redox balance, via reduction of oxidative stress, which may lead to higher viability.

Higher availability of lactate may lead to preferred lactate consumption as an alternative source for pyruvate instead of glucose. Lactate is heavily produced in the early stage of cell cultivation and the metabolic shift from lactate production to lactate consumption in cell culture, particularly in high density or ultra-high density cell culture, is at about day 3 of cultivation. From about day 5 lactate may be limited without supplementation, particularly in high density or ultra-high density cell culture. The high lactate consumption is believed to result in lower glycolytic input and hence lower TCA input. This affects the entire metabolism with lower ROS production. The combination of lactate and cysteine may be beneficial as they partly target the same effectors. Cysteine affects the glutathione antioxidant pathway with reduced ROS levels and lactate downregulates metabolism and hence ROS production.

The methods according to the invention are in vitro methods of culturing cells and involve the use of mammalian cell lines used for high expression of a product of interest, such as a heterologous protein or a recombinant virus. Thus, in one embodiment the mammalian cell is a mammalian cell line, preferably an immortalized cell line. Preferred examples of mammalian cells or mammalian cell lines are CHO cells (such as DG44 and K1), NSO cells, HEK293 cells (such as HEK293 cells, HEK293F and HEK293T cells) and BHK21 cells. Preferably the mammalian cells or mammalian cell lines are adapted to growth in suspension. In a preferred embodiment the mammalian cells or mammalian cell line is a CHO cell. In certain embodiments the mammalian cell is a HEK293 cell or a CHO cell or a HEK293 cell or a CHO cell derived cell, preferably the mammalian cell is a CHO cell or a CHO derived cell.

The term “mammalian cell” as used herein refers to mammalian cell lines suitable for the production of a product of interest, such as a heterologous protein or a recombinant virus and may also be referred to as “host cells”. The mammalian cells are preferably transformed and/or immortalized cell lines. They are adapted to serial passages in cell culture, preferably serum-free cell culture and/or preferably as suspension culture, and do not include primary non-transformed cells or cells that are part of an organ structure. Preferred mammalian cells for heterologous protein production are rodent cells such as hamster cells, particularly BHK21, BHK TK-, CHO, CHO-K1, CHO-DXB11 (also referred to as CHO-DUKX or DuxB11), a CHO-S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. These cells are particularly suitable for GS-based selection (such as methionine sulfoximine (MSX) selection) of clones stably expressing the heterologous protein. In one embodiment of the invention the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative thereof.

Preferred mammalian cells for recombinant virus production are hamster or human cells, particularly BHK21, BHK TK-, CHO (including CHO-K1, CHO-DXB11 (also referred to as CHO-DUKX or DuxB11, CHO-S and CHO-DG44) and HEK293 cells or the derivatives/progenies of any of such cell line. More preferably the mammalian cells for recombinant virus production are human cells, such as HEK293 cells and derivatives thereof (including HEK293F and HEK293T cells), preferably adapted for serum-free suspension culture.

The mammalian cell may further comprise one or more expression cassette(s) encoding a heterologous protein, such as a therapeutic protein, preferably a recombinant secreted therapeutic protein. The host cells may also be murine cells such as murine myeloma cells, such as NS0 and Sp2/0 cells or the derivatives/progenies of any of such cell line. Non-limiting examples of mammalian cells which can be used in the meaning of this invention are also summarized in Table 1. However, derivatives/progenies of those cells, other mammalian cells, including but not limited to human, mice, rat, monkey, and rodent cell lines, can also be used in the present invention, particularly for the production of biopharmaceutical proteins.

TABLE 1
Mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (═CHO duk; CHO/dhfr⁻; | |
| CHO-DXB11) | |
| CHO-DUKX 5A-HS-MYC | ATCC CRL-9010 |
| CHO-DG44 | Urlaub G, et al., 1983. Cell. |
| | 33: 405-412. |
| CHO Pro-5 | ATCC CRL-1781 |
| CHO-S | Life Technologies A1136401; |
| | CHO-S is derived from CHO |
| | variant Tobey et al. 1962 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J. et al., 2011. |
| | BMC Proc. 5(Suppl 8): P133. |
| PER.C6 ® | Pau et al., 2001. Vaccines. 19: 2716- |
| | 2721. |
| H4-II-E | ATCC CRL-1548 |
| | ECACC No. 87031301 |
| | Reuber, 1961. J. Natl. |
| | Cancer Inst. 26: 891-899. |
| | Pitot H C, et al., 1964. Natl. Cancer |
| | Inst. Monogr. 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Mammalian cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The term “heterologous protein” as used herein refers to any protein not naturally expressed by the mammalian cells and introduced into the mammalian using recombinant technology. Preferably a recombinant nucleic acid is introduced into the mammalian cells, such as by transfection or transduction. The nucleic acid may be stably integrated into the genome or transiently expressed. Preferably the nucleic acid encoding the heterologous protein is stably integrated into the genome. Preferred mammalian cell line for heterologous protein expression are CHO cells, such as CHO-DG44 and CHO-K1.

The heterologous protein may be any therapeutically relevant protein. Examples for therapeutic proteins are without being limited thereto antibodies, fusion proteins, cytokines and growth factor. The heterologous protein produced in the mammalian cells according to the methods of the invention includes but is not limited to an antibody or a fusion protein, such as a Fc-fusion proteins. Other heterologous proteins can be for example enzymes, cytokines, lymphokines, adhesion molecules, receptors and derivatives or fragments thereof, and any other polypeptides and scaffolds that can serve as agonists or antagonists and/or have therapeutic or diagnostic use.

A preferred heterologous protein is an antibody or a fragment or derivative thereof or a fusion protein. Thus, the method according to the invention can be advantageously used for production of antibodies, preferably monoclonal antibodies. Typically, an antibody is mono-specific, but an antibody may also be multi-specific. Thus, the method according to the invention may be used for the production of mono-specific antibodies, multi-specific antibodies, or fragments thereof, preferably of antibodies (mono-specific), bispecific antibodies, trispecific antibodies or fragments thereof, preferably antigen-binding fragments thereof. Unless specifically mentioned, the term “antibody” refers to a mono-specific antibody. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

The term “antibody”, “antibodies”, or “immunoglobulin(s)” as used herein relates to proteins selected from among the globulins, which are naturally formed as a reaction of the host organism to a foreign substance (=antigen) from differentiated B-lymphocytes (plasma cells). There are various classes of immunoglobulins: IgA, IgD, IgE, IgG, IgM, IgY, IgW. Preferably the antibody is an IgG antibody, more preferably an IgG1 or an IgG4 antibody. The terms immunoglobulin and antibody are used interchangeably herein. Antibody include monoclonal, monospecific and multi-specific (such as bispecific or trispecific) antibodies, a single chain antibody, an antigen-binding fragment of an antibody (e.g., a Fab or F(ab′)₂ fragment), a disulfide-linked Fv, etc. Antibodies can be of any species and include chimeric and humanized antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues. Antibodies may be produced through chemical synthesis, via recombinant or transgenic means, via cell (e.g., hybridoma) culture, or by other means.

Typically, antibodies are tetrameric polypeptides composed of two pairs of a heterodimer each formed by a heavy and light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or “region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

Antigen-binding fragments include without being limited thereto e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′)₂ fragments, which may be prepared by proteolytic cleavage with pepsin.

Using genetic engineering methods, it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulphide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies, such as bispecific or trispecific antibodies.

Preferred therapeutic antibodies according to the invention are multispecific antibodies, particularly bispecific or trispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be built on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

Another preferred therapeutic protein is a fusion protein, such as an Fc-fusion protein. Thus, the invention can be advantageously used for production of fusion proteins, such as Fc-fusion proteins. Furthermore, the method of increasing protein producing according to the invention can be advantageously used for production of fusion proteins, such as Fc-fusion proteins.

The effector part of the fusion protein can be the complete sequence or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgG, IgA, IgE, IgD or IgM. Preferentially they are derived from human immunoglobulin, more preferred from human IgG and even more preferred from human IgG1 and IgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising e.g. the N-linked glycosylation site.

In a further aspect a method of producing a product of interest is provided using the methods of the invention and further comprising a step of isolating and/or purifying the product or interest and optionally formulating the product of interest into a pharmaceutically acceptable formulation. In one embodiment the product of interest is a heterologous protein or a recombinant virus. Specifically, a method of producing a heterologous protein is provided using the methods of the invention and further comprising a step of isolating and/or purifying the heterologous protein and optionally formulating the heterologous protein into a pharmaceutically acceptable formulation. Alternatively, a method of producing a recombinant virus is provided using the methods of the invention and further comprising a step of isolating and/or purifying the recombinant virus and optionally formulating the recombinant virus into a pharmaceutically acceptable formulation, such as for vaccination or gene therapy.

The heterologous protein may be a therapeutic protein, especially the antibody, antibody fragment, antibody derivative or Fc-fusion protein is preferably recovered/isolated from the culture medium as a secreted polypeptide. It is necessary to purify the therapeutic protein from other recombinant proteins and host cell proteins to obtain substantially homogenous preparations of the heterologous protein. As a first step, cells and/or particulate cell debris are removed from the culture medium. Further, the heterologous protein is purified from contaminant soluble proteins, polypeptides and nucleic acids, for example, by fractionation on immunoaffinity or ion-exchange columns, ethanol precipitation, reverse phase HPLC, Sephadex chromatography, and chromatography on silica or on a cation exchange resin such as DEAE. Methods for purifying a heterologous protein expressed by mammalian cells are known in the art.

Preferably the heterologous protein is an antibody or an antigen-binding fragment thereof, a multispecific antibody, such as a bispecific antibody or trispecific, or a multispecific antigen-binding fragment thereof or a fusion protein. The antibody or the multispecific antibody (e.g. bispecific or trispecific antibody) may be an IgG1, IgG2a, IgG2b, IgG3 or IgG4 antibody, preferably an IgG1 or IgG4 antibody.

In another embodiment, the product of interest is a recombinant virus. The term “recombinant virus” as used herein refers to any virus produced using recombinant technology, particularly suitable for gene therapy or modification of cells for adoptive cell transfer. A recombinant virus may also express modified proteins and or proteins heterologous to the virus. Preferred recombinant viruses include, but are not limited to lentivirus, adenovirus, adeno-associated virus (AAV), herpes simplex virus, reovirus, Newcastle disease virus, measles virus, vaccinia virus, influence virus and vesicular stomatitis virus (VSV). Preferably the recombinant virus is an adeno-associated virus or a vesicular stomatitis virus. A preferred mammalian cells for the production of adeno-associated virus or vesicular stomatitis virus are HEK293 cells or derivatives thereof. For recombinant virus production, mammalian cells may be stably and/or transiently transfected to comprise the nucleic acid encoding the recombinant virus, or the mammalian cells may be transduced to comprise the nucleic acid encoding the recombinant virus, to efficiently produce the virus. For example, VSV may be produced by transduction of mammalian cells, such as HEK293 cells or derivatives thereof, in serum-free suspension culture. Thus, in one embodiment the invention relates to a method of producing a product of interest in a fed-batch process comprising: (a) providing mammalian cells comprising a nucleic acid encoding a recombinant virus; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the recombinant virus; and (e) optionally isolating the product of interest.

Step (a), providing mammalian cells comprising a nucleic acid encoding a recombinant virus, comprises transducing or transfecting the cells for introducing the nucleic acid encoding the recombinant virus, wherein transfection may be transient transfection, stable transfection or a combination thereof and wherein the transfection may involve co-transfection of multiple nucleic acid molecules, such as plasmids. While for stably transfected cells comprising a nucleic acid encoding a recombinant virus, the mammalian cell comprising the nucleic acid encoding the recombinant virus (as for stably transfected mammalian cells encoding a heterologous protein) is inoculated in a basal medium to provide a cell culture, transient transfection or transduction may occur following inoculation of the mammalian cell inoculated in a basal medium to provide a cell culture and even after feeding started. The mammalian cell may be transiently transfected with one or more nucleic acid molecule encoding the recombinant virus or transduced with the recombinant virus at a desired cell density and feeding may start shortly after starting the cell culture on day 0 or more typically one, two or three days after starting the culture, which may be before or after transfecting or transducing the mammalian cell. In a preferred embodiment, the cells are transiently transfected with one or more nucleic acid molecules encoding the recombinant virus or transduced with the recombinant virus at the desired cell density at cell inoculation or after a certain period of time when the desired cell density is achieved, which may be after the feeding has started, such as at days 1-7 after starting the culture, preferably at days 2-5 after starting the culture, more preferably at days 3-5 after starting the culture. Preferably, HEK293 cells or derivatives thereof (such as HEK293F or HEK293T cells) are transduced with the recombinant virus (such as VSV) following inoculation and optionally feeding to provide the HEK293 cells or derivatives thereof comprising a nucleic acid encoding the recombinant virus (such as VSV). Methods for producing recombinant virus in suspension serum-free cell culture, such as VSV, are per se known in the art, e.g., from Elahi S. M. et al., (Journal of Biotechnology (2019) 289:114-149).

In certain other embodiments of the methods according to the invention the nucleic acid encodes a heterologous protein and the product titers and/or cell specific productivity is increased compared to product titers and/or cell specific productivity of the heterologous protein produced by the same method, wherein the feed medium adds cysteine at or below 0.19 mM/day in the absence of lactate, preferably wherein the feed medium adds cysteine below 0.225 mM/day in the absence of lactate. In one embodiment the product titer and/or cell specific productivity is increased by at least 20%, at least 40%, at least 50%, at least 60% at least 80%, at least 90%, at least 100% or more than 100%. In one embodiment the heterologous protein is an antibody or an antigen-binding fragment thereof, a bispecific antibody, a trispecific antibody or a fusion protein.

In further separate or additional embodiment the nucleic acid encodes a heterologous protein and the relative amount of high mannose structures in a population of the heterologous protein is reduced compared to a population of the heterologous protein produced by the same method, wherein the feed medium adds cysteine at or below 0.19 mM/day in the absence of lactate, preferably wherein the feed medium adds cysteine below 0.225 mM/day in the absence of lactate. In one embodiment the relative amount of high mannose structures in a population of the heterologous protein may be reduced by at least 20%, at least 40%, at least 50%, at least 60% at least 80%, or at least 90%. High mannose structures may be mannose 5, mannose 6, mannose 7, mannose 8 and/or mannose 9 structures. Preferably the reduced relative amount of high mannose structures in a population of the heterologous protein is the reduced relative amount of mannose 5 structures in a population of the heterologous protein. In a preferred embodiment the heterologous protein is an antibody. More preferably the relative amount (of total) of the population of the antibody having mannose 5 structures is less than 20%, preferably less than 10%, more preferably less than 5%. The term “population of the heterologous protein” as used herein refers to all heterologous proteins in a sample encoded by the same nucleic acid. A population of heterologous proteins may be heterogeneous, e.g., with regard to the glycosylation or post-translational modifications or degradation of the individual heterologous proteins in the population.

In a further separate or additional embodiment the nucleic acid encodes a heterologous protein and the relative amount (of total) of acidic species in a population of the heterologous protein is reduced compared to a population of the heterologous protein produced by the same method, wherein the feed medium adds the same concentration of cysteine in the absence of lactate. The relative amount of acidic species in a population of the heterologous protein may be reduced by at least 20%, at least 40%, at least 50%, at least 60% at least 80%, or at least 90%. In one embodiment the heterologous protein is selected from the group consisting of an antibody or an antigen-binding fragment thereof, a bispecific antibody, a trispecific antibody or a fusion protein. In a preferred embodiment the heterologous protein is an antibody (monospecific, bispecific or trispecific) or an antigen-binding fragment thereof.

In another embodiment of the methods according to the invention the nucleic acid encodes recombinant virus and the virus titer is increased compared to a virus titer produced by the same method, wherein the feed medium adds cysteine at or below 0.19 mM/day in the absence of lactate, preferably wherein the feed medium adds cysteine below 0.225 mM/day in the absence of lactate. In one embodiment the virus titer is increased by at least 20%, at least 40%, at least 50%, at least 60% at least 80%, at least 90%, at least 100% or more than 100%.

In certain embodiments of the methods according to the invention the basal medium is a serum-free and chemically defined medium and the feed medium is a serum-free and chemically defined medium. Moreover, the basal medium and the feed medium may be protein-free.

Also provided is a method of reducing acidic species in a heterologous protein produced in a fed-batch process comprising: (a) providing mammalian cells comprising a nucleic acid encoding the heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount (of total) of acidic species in a population of the heterologous protein is reduced compared to a population of the heterologous protein produced by the same method wherein the feed medium adds the same concentration of cysteine in the absence of lactate. In one embodiment the heterologous protein is selected from the group consisting of an antibody or an antigen-binding fragment thereof, a bispecific antibody, a trispecific antibody or a fusion protein. Preferably the heterologous protein is an antibody (wherein the antibody may be a monospecific or multispecific antibody) or an antigen-binding fragment thereof.

The relative amount of acidic species in a population of the heterologous protein may be reduced by at least 20%, at least 40%, at least 50%, at least 60% at least 80%, or at least 90%. Preferably less than 50% of the heterologous protein in the population is an acidic species, more preferably less than 40%, less than 30%, less than 20%, and even more preferably less than 10% of the heterologous protein in the population is an acidic species.

The term “acidic species” as used herein refers to acidic charge variants of a heterologous protein, particularly of a recombinant monoclonal antibody produced by post-translational modifications. Acidic species are typically collected using cation or anion exchange chromatography, such as (WCX)-10 and may be characterized by LC-MS. Acidic charge variants include, but are not limited to methionine oxidation, asparagine deamination, cysteinylation, glycation, reduced disulfide bonds.

Also provided is a method of reducing high mannose structures in a heterologous protein produced in a fed-batch process comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount of high mannose structures in a population of the heterologous protein is reduced compared to a population of the heterologous protein produced by the same method wherein the feed medium adds cysteine at or below 0.19 mM/day in the absence of lactate, preferably wherein the feed medium adds cysteine below 0.225 mM/day in the absence of lactate. In a preferred embodiment the high mannose structure is a mannose 5 structure. In one embodiment the heterologous protein is selected from the group consisting of an antibody or an antigen-binding fragment thereof, a bispecific antibody, a trispecific antibody or a fusion protein. Preferably the heterologous protein is an antibody (wherein the antibody may be a monospecific or multispecific antibody) or an antigen-binding fragment thereof. Also provided herein is a heterologous protein produced by the methods according to the invention, particularly by the method of reducing high mannose structures in a heterologous protein according to the invention and by the method of reducing acidic species in a heterologous protein according to the invention.

The term “high mannose structure” as used herein refers to high-mannose N-linked glycans containing unsubstituted terminal mannose sugars. These glycans typically contain between five and nine mannose residues attached to the chitobiose (GIcNAc₂) core and hence include Man₆GlcNAc₂, Man₆GlcNAc₂, Man₇GlcNAc₂, Man₈GlcNAc₂ and Man₉GlcNAc₂ glycans, also referred to mannose 5 structures (Man-5), mannose 6 structures (Man-6), mannose 7 structures (Man-7), mannose 8 structures (Man-8) and mannose 9 structures (Man-9), respectively. High mannose structures are associated with a short half-life of the heterologous protein and mannose 5 structures are considered to be representative for high mannose structures and typically determined to access high mannose structures. Thus, the high mannose structure is preferably a mannose 5 structure.

The term isolating the product of interest” as used herein includes isolating the cell culture medium comprising the product of interest from the mammalian cells, and/or purifying the product of interest from the cell culture medium following harvest of the cell culture medium comprising the product of interest, and/or lysing the cells and purifying the product of interest from the mammalian cell lysate. Likewise the term “isolating the heterologous protein” or “isolating the antibody” or “isolating the recombinant virus” as used herein includes isolating the cell culture medium comprising the heterologous protein and/or antibody or recombinant virus from the mammalian cells, and/or purifying the heterologous protein and/or antibody or recombinant virus from the cell culture medium following harvest of the cell culture medium comprising the heterologous protein and/or antibody or recombinant virus, and/or lysing the cells and purifying the heterologous protein and/or antibody or recombinant virus from the mammalian cell lysate. Methods for purifying heterologous proteins, including antibodies, or recombinant virus are known in the art.

Also provided is a method of preventing negative effects of cysteine on product quality characteristics when producing a heterologous protein in a fed-batch process comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the cell culture medium comprising the heterologous protein from the mammalian cells; wherein the negative effects on product quality characteristics are in a population of the heterologous protein are reduced compared to a population of the heterologous protein produced by the same method wherein the feed medium adds the same concentration of cysteine in the absence of lactate. In one embodiment the heterologous protein is selected from the group consisting of an antibody or an antigen-binding fragment thereof, a bispecific antibody, a trispecific antibody or a fusion protein. Preferably the heterologous protein is an antibody (including a monospecific, a bispecific antibody or a trispecific antibody) or a fragment thereof. The negative effects on product quality characteristics may be, without being limited thereto, high or increased high mannose structures (preferably high mannose 5 structures), high or increased low molecular weight species and/or high or increased acidic species.

Process optimization is particularly relevant for high seeded bioprocesses. Higher seeding density minimizes the unproductive exponential growth phase and leads to a comparatively short, high-productive process. CHO cells are most commonly used for biopharmaceutical production of heterologous proteins, such as antibodies or fusion proteins in fed-batch processes. Those processes consist of an unproductive or less productive growth phase in the beginning, where cells accumulate in the bioreactor, followed by a stationary phase in which most of the product is generated. The length of the growth phase directly affects process duration and volumetric productivity. By usage of a perfusion system in the N-1 seed train, this growth phase is shifted to the N-1 bioreactor. Perfusion mode allows continuous removal of waste metabolites and addition of nutrients by continuous media exchange in order to reach high cell densities up to 100×10⁶ cells per mL with acceptable viability. Through application of a perfusion process in the N-1 stage, high seeding densities can be achieved in the subsequent N-stage (i.e., the fed-batch process), resulting in an immediate high productivity. Ultra-High Seed Density (uHSD) fed-batch processes can produce the same amount of titer with comparable product quality in a shorter period of time, which increases the manufacturing capacity. Furthermore, they can produce a higher amount of final product titer in the same time period than lower seeded processes. Thus, high seeding density cultures are promising for improving overall productivity, but are also particularly demanding and sensitive with regard to medium optimization. In order to improve ultra-high seeding density processes the inventors found that cysteine and/or lactate has a beneficial effect on culture performance. Surprisingly it was found that this also improves cell cultures using normal seeding densities.

According to the invention the cysteine is added at 0.225 mM/day or higher. Particularly for high density seeding processes or ultrahigh density seeding processes it may be beneficial to add cysteine at 0.3 mM/day or higher, at 0.4 mM/day or higher, or at 0.5 mM/day or higher. In one embodiment the cysteine is added from about 0.3 mM/day to about 0.6 mM/day, or from about 0.4 mM/day to about 0.6 mM/day. An increase or decrease according to the invention may be determined compared to a method or culture, wherein the feed medium adds cysteine at or below 0.19 mM/day in the absence of lactate, preferably wherein the feed medium adds cysteine below 0.225 mM/day in the absence of lactate. In case cysteine is added at 0.3 mM/day or higher in a high density seeding processes or ultrahigh density seeding processes, in one embodiment an increase or decrease is determined compared to a method or culture, wherein the feed medium adds cysteine at or below 0.28 mM/day in the absence of lactate.

However, uHSD processes are often characterized by an early viability drop. The addition of lactate and cysteine according to the methods of the invention overcomes these problems. Hence in one embodiment the fed-batch process is a uHSD fed-batch process.

### Use of Lactate and Cysteine in a Feed Medium to Improve Cell Culture Performance

In one aspect the invention relates to a use of lactate in a feed medium for reducing acidic species in a heterologous protein produced in a fed-batch process, wherein the feed medium comprises cysteine. Preferably the feed medium provides cysteine at 0.225 mM/day or higher. In one embodiment of the uses of the invention the group consisting of an antibody or an antigen-binding fragment thereof, a bispecific antibody, a trispecific antibody or a fusion protein. Preferably the heterologous protein is an antibody (wherein the antibody may be monospecific and multispecific).

The invention also relates to the use of lactate in a feed medium for reducing high mannose structures, such as mannose 5 structures in a heterologous protein produced in a fed-batch process, wherein the feed medium comprises cysteine. Preferably the feed medium provides cysteine at 0.225 mM/day or higher.

The invention also relates to the use in a feed medium for preventing negative effects of cysteine on product quality characteristics of a heterologous protein, preferably wherein the negative effects on product quality characteristics are increased high mannose structures (such as mannose 5 structures), increased low molecular weight species and/or increased acidic species. In one embodiment the feed medium provides cysteine at 0.225 mM/day or higher, such as at 0.25 mM/day or higher, at 0.3 mM/day or higher, or at 0.4 mM/day or higher.

The invention also relates to the use of lactate and cysteine in a feed medium for increasing heterologous protein titer and/or cell-specific productivity in a fed-batch process. Also provided is the use of lactate and cysteine in a feed medium for increasing recombinant virus production in a fed-batch process.

In one embodiment of the uses of the invention the heterologous protein is selected from the group consisting of an antibody or an antigen-binding fragment thereof, a bispecific antibody, a trispecific antibody or a fusion protein. Preferably the heterologous protein is an antibody (wherein the antibody may be monospecific and multispecific). The fed-batch process according to the uses according to the invention comprises culturing a mammalian cell, wherein the mammalian cell may be any mammalian cell as described herein, preferably, the mammalian cell is a HEK293 cell or a CHO cell or a HEK293 cell or CHO cell derived cell, preferably the mammalian cell is a CHO cell or a CHO derived cell.

The use of lactate or lactate and cysteine is according to the methods of the invention. Thus, the lactate and cysteine are to be added at a lactate/cysteine molar ratio of about 8:1 to 50:1, about 10:1 to 50:1, preferably about 10:1 to about 30:1, more preferably about 15:1 to about 30:1 and even more preferably about 15:1 to about 30:1. In one embodiment, the lactate is added at 3 mmol/L/day or higher, 3.8 mmol/L/day or higher, at 5 mmol/L/day or higher, preferably at 7 mmol/L/day lactate or higher, at 7.8 mmol/L/day or higher, at 10 mmol/L/day or higher or even at 15 mmol/L/day or higher. The lactate in the cell culture medium may be maintained at 0.5 g/L or higher, at 1 g/L or higher, preferably at 2 g/L or higher, preferably between 2 and 4 g/L, more preferably between about 2 and 3 g/L. More specifically, the lactate concentration in the cell culture medium is maintained between about 1 g/L and about 4.5 g/L (˜10 to 50 mM), between about 2 g/L and about 4 g/L (˜20-45 mM), and preferably between about 2 g/L and about 3 g/L (˜20-35 mM). The lactate (MW=89.07 g/mol) may be provided as a salt and/or a hydrate thereof and/or as lactic acid, preferably as sodium lactate (MW=112.06 g/mol), wherein 1 g/L of lactate equates to about 1.25 g/L of sodium lactate. The salt and/or hydrate of lactate or the lactic acid is provided at an equimolar concentration to the lactate concentration provided herein. In a preferred embodiment the basal medium does not contain lactate added as a medium component. However, lactate may be generated during culture in the basal medium as a metabolite.

The cysteine may be provided as cysteine or a salt and/or a hydrate thereof, cystine or a salt thereof or a dipeptide or tripeptide comprising cysteine. The cysteine salt and/or hydrate or the cystine or the salt thereof or the dipeptide or tripeptide comprising cysteine is provided at an equimolar concentration to the cysteine concentrations provided herein. According to the invention the cysteine is added at 0.225 mM/day or higher. Wherein the cysteine is added to the basal medium resulting in the cell culture medium or to the resulting cell culture medium. Preferably the cysteine is added at 0.25 mM/day or higher, 0.3 mM/day or higher, more preferably at 0.4 mM/day or higher, more preferably at 0.5 mM/day or higher. In one embodiment the cysteine is added from about 0.225 mM/day to about 0.6 mM/day, from about 0.25 mM/day to about 0.6 mM/day from about 0.3 mM/day to about 0.6 mM/day, or from about 0.4 mM/day to about 0.6 mM/day.

In certain embodiments of the uses according to the invention the heterologous protein product titer and/or cell specific productivity is increased compared to product titer and/or cell specific productivity of the heterologous protein produced by the same method, wherein the feed medium adds cysteine at or below 0.19 mM/day in the absence of lactate, preferably wherein the feed medium adds cysteine below 0.225 mM/day in the absence of lactate. In one embodiment the product titer and/or cell specific productivity is increased by at least 20%, at least 40%, at least 50%, at least 60% at least 80%, at least 90%, at least 100% or more than 100%.

In another embodiment the relative amount of high mannose structures in a population of the heterologous protein may be reduced by at least 20%, at least 40%, at least 50%, at least 60% at least 80%, or at least 90%. Wherein reduced means compared to a population of the heterologous protein produced by the same method, wherein the feed medium adds cysteine at or below 0.19 mM/day in the absence of lactate, preferably wherein the feed medium adds cysteine below 0.225 mM/day in the absence of lactate. High mannose structures may be mannose 5, mannose 6, mannose 7, mannose 8 and/or mannose 9 structures. Preferably the reduced relative amount of high mannose structures in a population of the heterologous protein is the reduced relative amount of mannose 5 structures in a population of the heterologous protein. In a preferred embodiment the heterologous protein is an antibody and the relative amount (of total) of the population of the antibody having mannose 5 structures is less than 20%, preferably less than 10%, more preferably less than 5% or even less than 2%.

In a further separate or additional embodiment the heterologous protein may be selected from the group consisting of an antibody or an antigen-binding fragment thereof, a bispecific antibody, a trispecific antibody or a fusion protein, and the relative amount (of total) of acidic species in a population of the heterologous protein is reduced compared to a population of the antibody produced by the same method, wherein the feed medium adds the same concentration of cysteine in the absence of lactate. The relative amount of acidic species in a population of the antibody may be reduced by at least 20%, at least 40%, at least 50%, at least 60% at least 80%, or at least 90%. Preferably, the heterologous protein is an antibody (wherein the antibody may be a monospecific or multispecific antibody).

In another embodiment of the uses according to the invention a recombinant virus is produced and the virus titer is increased compared to a virus titer produced by the same method, wherein the feed medium adds cysteine at or below 0.19 mM/day in the absence of lactate, preferably wherein the feed medium adds cysteine below 0.225 mM/day in the absence of lactate. In one embodiment the virus titer is increased by at least 20%, at least 40%, at least 50%, at least 60% at least 80%, at least 90%, at least 100% or more than 100%.

The uses according to the invention may also be used in the high seed density and Ultra-High Seed Density (uHSD) fed-batch processes as described herein.

### A Feed Medium for Improved Cell Culture Performance

In yet another aspect, the invention relates to a feed medium for mammalian cell fed-batch culture comprising lactate and cysteine at a molar ratio (mM/mM) of lactate/cysteine of about 8:1 to about 50:1, wherein the cysteine is added at 0.225 mM/day or higher. Preferably the feed medium comprises one or more feed supplements for separate addition. Particularly the cysteine may be added in a two-feed strategy, such as adding a feed medium comprising cysteine and a feed supplement added separately comprising cysteine. Lactate is preferably added with the feed medium, but may also be added separately in a feed supplement. In certain embodiments the feed medium or the feed supplement is chemically defined (optionally comprising a recombinant protein, such as insulin or insuline like growth factor (IGF)). Moreover, the feed medium or the feed supplement according to the invention does not contain cells (i.e., is not a cell culture comprising cells), has not been in contact with cells in culture (a pre-conditioned medium derived from a cell culture) and/or does not contain cell derived metabolic waste products. The feed medium according to the invention may be used in the methods and uses described herein and hence the embodiments specified and described for the methods likewise apply to the feed medium.

The feed medium may also be provided in a kit. Thus, the invention also relates to a kit comprising (a) a concentrated feed medium for mammalian cell fed-batch culture comprising lactate and optionally cysteine, and (b) an aqueous supplement separate from the concentrated feed medium comprising cysteine, wherein the feed medium and the supplement provide a lactate/cysteine molar ratio (mM/mM) of about 8:1 to about 50:1 and cysteine at 0.225 mM/day or higher in a daily addition of less than 5%, preferably less than 4%, more preferably less than 3.5% of the cell culture starting volume. The feed medium provided by the kit according to the invention may be used in the methods and uses described herein and hence the embodiments specified and described for the methods likewise apply to the kit.

Particularly the feed medium and the kit is particularly useful for a fed-batch process comprising culturing a mammalian cell, wherein the mammalian cell may be any mammalian cell as described herein, preferably, the mammalian cell is a HEK293 cell or a CHO cell or a HEK293 cell or CHO cell derived cell, preferably the mammalian cell is a CHO cell or a CHO derived cell.

The feed medium is adapted to provide lactate and cysteine according to the methods of the invention. Thus, feed medium or the kit providing the feed medium is adapted to provide lactate and cysteine at a lactate/cysteine molar ratio of about 8:1 to 50:1, about 10:1 to 50:1, preferably about 10:1 to about 30:1, more preferably about 15:1 to about 30:1 and even more preferably about 15:1 to about 25:1. In one embodiment, the lactate is provided at 3 mmol/L/day or higher, at 3.8 mmol/L/day or higher, at 5 mmol/L/day or higher, preferably at 7 mmol/L/day lactate or higher, at 7.8 mmol/L/day or higher, at 10 mmol/L/day or higher or even at 15 mmol/L/day or higher. The lactate (MW=89.07 g/mol) may be provided as a salt, an ester and/or a hydrate thereof and/or as lactic acid, preferably a salt, such as sodium lactate (MW=112.06 g/mol), wherein 1 g/L of lactate equates to about 1.25 g/L of sodium lactate. The salt and/or hydrate of lactate or the lactic acid is provided at an equimolar concentration to the lactate concentration provided herein.

The cysteine may be provided in the feed medium or the kit according to the invention as cysteine or a salt and/or a hydrate thereof, cystine or a salt thereof or a dipeptide or tripeptide comprising cysteine. The cysteine salt and/or hydrate or the cystine or salts thereof or the dipeptide or tripeptide comprising cysteine is provided at an equimolar concentration to the cysteine concentrations provided herein. The feed medium or kit is adapted to provide the cysteine at 0.225 mM/day or higher to the basal medium or the cell culture medium. Preferably the cysteine is added at 0.25 mM/day or higher, at 0.3 mM/day or higher, more preferably at 0.4 mM/day or higher, more preferably at 0.5 mM/day or higher. In one embodiment the cysteine is added from about 0.225 mM/day to about 0.6 mM/day, from about 0.25 mM/day to about 0.6 mM/day, from about 0.3 mM/day to about 0.6 mM/day, or from about 0.4 mM/day to about 0.6 mM/day.

The feed medium and the kit according to the invention may also be used in the high seed density and Ultra-High Seed Density (uHSD) fed-batch processes as described herein.

In view of the above, it will be appreciated that the invention also encompasses the following items:

Item 1 provides a method of producing a product of interest in a fed-batch process comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the product of interest; and (e) optionally isolating the product of interest.

Item 2 provides the method according to item 1, wherein the feed medium is added daily, preferably continuously.

Item 3 provides the method according to item 1 or 2, wherein the molar ratio of lactate/cysteine is about 10:1 to 50:1, preferably about 10:1 to about 30:1.

Item 4 provides the method according to any one of items 1-3, wherein the lactate is added at 3 mmol/L/day or higher, at 5 mmol/L/day or higher, at 7 mmol/L/day or higher, or at 10 mmol/L/day or higher.

Item 5 provides the method according to item 4, wherein the lactate in the cell culture medium is maintained at 0.5 g/L or higher, 1 g/L or higher, 2 g/L or higher, preferably between 2 and 4 g/L

Item 6 provides the method according to any one of items 1 to 5, wherein the cysteine (a) is provided as cysteine or a salt and/or hydrate thereof, cystine or a salt thereof or a dipeptide or tripeptide comprising cysteine; and/or (b) the cysteine is added at 0.25 mM/day or higher, at 0.3 mM/day or higher, or at 0.4 mM/day or higher.

Item 7 provides the method according to any one of items 1 to 6, wherein the product of interest is a heterologous protein or a recombinant virus.

Item 8 provides the method according to any one of items 1 to 7, wherein the nucleic acid encodes a heterologous protein and the product titers and/or cell specific productivity is increased compared to the product titers and/or cell specific productivity of the heterologous protein produced by the same method, wherein the feed medium adds cysteine at or below 0.19 mM/day in the absence of lactate.

Item 9 provides the method according to any one of items 1 to 8, wherein the nucleic acid encodes a heterologous protein and the relative amount of high mannose structures in a population of the heterologous protein is reduced compared to a population of the heterologous protein produced by the same method, wherein the feed medium adds cysteine at or below 0.19 mM/day in the absence of lactate, preferably wherein the high mannose structures are mannose 5 structures.

Item 10 provides the method according to any one of items 1 to 9, wherein the nucleic acid encodes a heterologous protein and wherein the relative amount (of total) of acidic species in a population of the heterologous protein is reduced compared to a population of the heterologous protein produced by the same method, wherein the feed medium adds the same concentration of cysteine in the absence of lactate.

Item 11 provides the method of any one of items 1 to 10, wherein the basal medium and the feed medium is serum-free and chemically defined.

Item 12 provides the method of any one of items 1 to 11, wherein the heterologous protein is an antibody or an antigen-binding fragment thereof, a bispecific antibody, a trispecific antibody or a fusion protein.

Item 13 provides the method of item 12, wherein the antibody, the bispecific antibody or the trispecific antibody is an IgG1, IgG2a, IgG2b, IgG3 or IgG4 antibody.

Item 14 provides a method of culturing mammalian cells in a fed-batch process comprising: (a) providing mammalian cells comprising a nucleic acid encoding a product of interest; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; and (d) culturing the mammalian cells in the cell culture medium under conditions that allow expression of the product of interest.

Item 15 provides a method of reducing acidic species in a heterologous protein produced in a fed-batch process comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount of acidic species in a population of the heterologous protein is reduced compared to a population of the heterologous protein produced by the same method wherein the feed medium adds the same concentration of cysteine in the absence of lactate.

Item 16 provides a method of reducing high mannose structures in a heterologous protein produced in a fed-batch process comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein; wherein the relative amount of the high mannose structures in a population of the heterologous protein is reduced compared to a population of the heterologous protein produced by the same method wherein the feed medium adds the cysteine at or below 0.19 mM/day in the absence of lactate, preferably wherein the high mannose structures are mannose 5 structures.

Item 17 provides a method of preventing negative effects of cysteine on product quality characteristics when producing a heterologous protein in a fed-batch process comprising: (a) providing mammalian cells comprising a nucleic acid encoding a heterologous protein; (b) inoculating the mammalian cells in a basal medium to provide a cell culture; (c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; (d) culturing the mammalian cells in the cell culture medium under conditions that allow expression of the heterologous protein; and (e) optionally isolating the heterologous protein from the mammalian cells; wherein the negative effects on product quality characteristics in a population of the heterologous protein are reduced compared to a population of the heterologous protein produced by the same method wherein the feed medium adds the same concentration of cysteine in the absence of lactate.

Item 18 provides the method of any one of items 1-17, wherein the mammalian cell is a HEK293 cell or a CHO cell or a HEK293 cell or a CHO cell derived cell, preferably the mammalian cell is a CHO cell or a CHO derived cell.

Item 19 provides a heterologous protein produced by the method of any one of items 15 to 17.

Item 20 provides a use of lactate in a feed medium for reducing acidic species in a heterologous protein produced in a fed-batch process, wherein the feed medium adds cysteine at 0.225 mM/day or higher.

Item 21 provides a use of lactate in a feed medium for reducing high mannose structures in a heterologous protein produced in a fed-batch process, wherein the feed medium comprises cysteine at 0.225 mM/day or higher, preferably wherein the high mannose structures are mannose 5 structures.

Item 22 provides a use of lactate in a feed medium for preventing negative effects of cysteine on product quality characteristics of a heterologous protein produced in a fed-batch process, preferably wherein the negative effects on product quality characteristics are increased high mannose structures, increased low molecular weight species and/or increased acidic species.

Item 23 provides a use of lactate and cysteine in a feed medium for increasing heterologous protein titer and/or cell-specific productivity in a fed-batch process.

Item 24 provides the use of any one of items 20 to 23, wherein the fed-batch process comprises culturing a mammalian cell, wherein the mammalian cell is a HEK293 cell or a CHO cell or a HEK293 cell or CHO cell derived cell, preferably the mammalian cell is a CHO cell or a CHO derived cell

Item 25 provides a feed medium for mammalian cell fed-batch culture comprising lactate and cysteine at a molar ratio (mM/mM) of lactate/cysteine of about 8:1 to about 50:1.

Item 26 provides the feed medium of item 25, wherein the feed medium comprises one or more feed supplements for separate addition.

Item 27 provides a kit comprising (a) a concentrated feed medium for mammalian cell fed-batch culture comprising lactate and optionally cysteine, and (b) an aqueous supplement separate from the concentrated feed medium comprising cysteine, wherein the feed medium and the supplement provide a lactate/cysteine molar ratio (mM/mM) of about 8:1 to about 50:1 and cysteine at 0.225 mM/day or higher in a daily addition of less than 5%, preferably less than 3.5% of the cell culture starting volume.

### EXAMPLES

### Example 1: uHSD (Ultra-High Seeding Density) Processes

A chinese hamster ovary (CHO) cell line (cell line A; CHO-K1 GS) producing a monoclonal IgG1 antibody (mAb) was cultivated in a 3L glass bioreactor system in fed-batch mode. The seed train cultures were processed in shake flasks until the N-1 stage which was processed in 2 L single-use bioreactor systems in a perfusion mode. The seeding cell densities were set at 10×10E06 cells/mL with a start volume of 2.2 L in a proprietor basal medium comprising 0.4 g/L cysteine HCl H₂O (2.3 mM cysteine; MW cysteine HCl H₂O=173.63 g/mol), 0.028 g/L L-cystine 2HCl (0.36 mM cysteine; MW cystine HCl=157.62 mM) and no lactate. The fed-batch cultivations were conducted for 13-14 days. Feed media (comprising 1.21 g/L cysteine HCl H₂O; 0.0069 M cysteine and no lactose) were added continuously from day 0 until the end of the process (d0-d9: 45 ml/L/day; d9-d14: 25 ml/L/day) and glucose was added to the process on demand and was maintained at 3 g/L to 5 g/L. Sodium lactate and additional cysteine HCl H₂O were added as bolus additions, as shown in Table 2. Lactate was added to the bioprocess uHSD LAC and uHSD LAC/CYS by bolus addition from day 3 to 13, if lactate concentration dropped under 2 g/L with a target concentration of 3 g/L (stock solution 238.5 g/L; 2.68 mol/l). Cysteine bolus additions to the processes uHSD LAC/CYS and uHSD CYS were performed from day 1 to 5 in a volume of 7 ml (stock solution cysteine HCl H₂O: 30 g/L (20.69 g/L cysteine; 0.17 mol/L). Cultivation samples were taken every 24 hours and cell counting and cell viability determination was performed using a Cedex HiRes analyzer (Roche, Germany). Glucose and lactate were determined using a Konelab Prime60i (Thermo Scientific, USA). The antibody concentration was determined with a Protein-A HPLC method (Thermo Scientific, USA).

TABLE 2
Experimental set ups tested with the uHSD processes

| | | Bolus addition | Bolus addition |
|---|---|---|---|
| Experiment | Number of | of sodium lactate | of cysteine (CYS HCL |
| ID | replicates | (300 g/L, 2.68 mol/l) | H₂O 30 g/L) |
| uHSD | 6 | — | — |
| uHSD LAC | 3 | day 3 to 13 if <2 | — |
| | | g/L to 3 g/L | |
| uHSD | 2 | day 3 to 13 if <2 | 7 ml daily (day 1-5) |
| LAC/CYS | | g/L to 3 g/L | |
| uHSD CYS | 1 | — | 7 ml daily (day 1-5) |

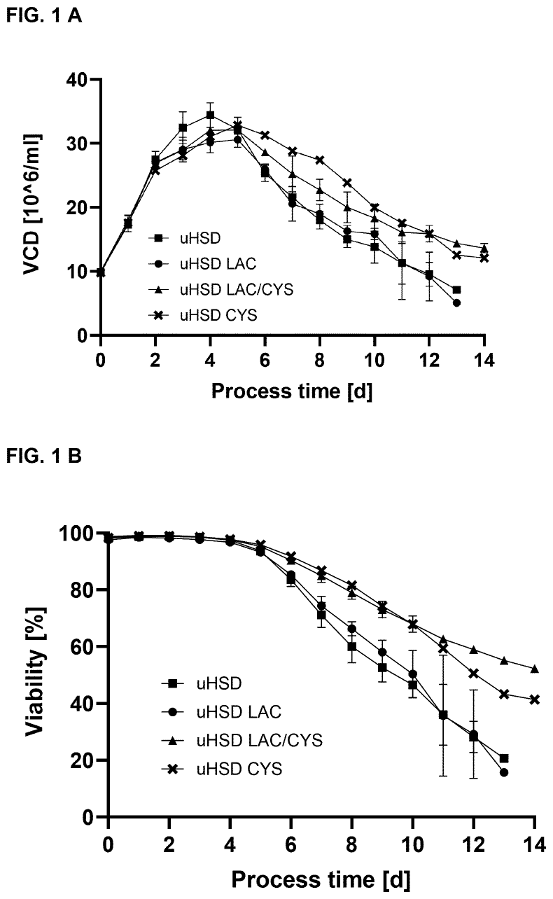

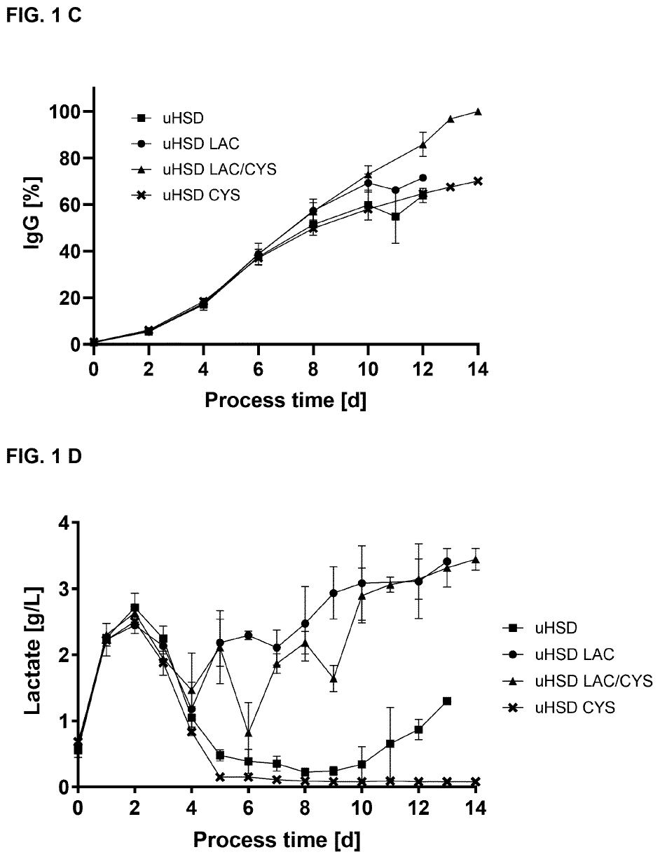

The effect of lactose, cysteine or lactose and cysteine on viable cell density (VCD), viability, product concentration and lactate concentrations are shown in FIG. 1A-D.

As may be taken from FIGS. 1 A and B feeding with lactate and cysteine (uHSD LAC/CYS) and cysteine alone (uHSD CYS) improved viability and VCD starting from about day 6 compared to cells fed without additional lactate and cysteine feed (uHSD) and fed with lactate alone (uHSD LAC). Particularly towards the end of the culture viability of cells fed with lactate and cysteine (uHSD LAC/CYS) seems to be even higher than for cells fed with cysteine alone (uHSD CYS).

With regard to production, lactate feeding (uHSD LAC) had a positive effect on IgG titer during cultivation, but it seems that this could not be sustained until the end of the culture. IgG titer in cell cultures obtaining an additional cys feed (uHSD CYS) were comparable to control cell culture (uHSD). Surprisingly IgG titer in cell cultures comprising lactate and cysteine (uHSD LAC/CYS cells) was strongly increased compared to bolus feeds with lactate or cysteine alone. This was mainly due to an increased specific productivity (pg/cell/day) following day 10 (data not shown).

As shown in FIG. 1D, lactate is depleted in the cultures between about days 5 and day 10 and was continuously above 2 g/L in the uHSD LAC/CYS cultures. The drop in lactate in uHSD CYS cultures at days 6 and 9 is likely to be due to the high cell concentration and a high specific lactate uptake rate.

Overall for high density feeding, the uHSD processes with a bolus addition of sodium lactate and cysteine resulted in an improved product titer and cell viability profile.

### Example 2: DoE Optimization Study in Regular Fed-Batch Processes

To further analyse the effect of cysteine and/or lactate in more detail on cell culture we performed regular fed-batch processes using regular seeding densities with two different cell lines under controlled conditions using a bioreactor.

Two CHO-K1 GS cell lines (cell line A and B) producing an IgG1 monoclonal antibody (mAb), respectively, were cultivated in an ambr 250 bioreactor system. The experiments were part of a Design of Experiments (DoE) study. The seed train cultures were processes in shake flasks and the seeding cell densities were set at 0.7×10E06 cells/ml. The fed-batch cultivations were conducted for 14 days. In contrast to the uHSD processes a continuous application of lactate and cystine was applied in these experiments in order to reduce high concentrations of the reactive compound cysteine in the bioreactor and in order to include lactate in the regular applied feed.

Feed media (comprising 1.1 g/L cysteine HCl H₂O; 0.0063 M cysteine) were added continuously from day 2 at 30 ml/L/day (of the culture starting volume) until the end of the processes and glucose was added to the processes on demand. Cultivation samples were taken every 24 hours and cell counting and cell viability determination was performed using a Cedex HiRes analyzer (Roche, Germany). Glucose and lactate were determined using a Konelab Prime60i (Thermo Scientific, USA). The antibody concentration was determined with a Protein-A HPLC method (Thermo Scientific, USA). Charge variants were analyzed using a PrpPac WCX-10 analytical column connected to a HPLC system with UV detection. Size variants were analyzed with a BEH200 SEC column connected to a HPLC system with UV detection. N-glycan determination was performed using a LabChip GXII and HT Glycan Reagent Kit.

To identify the interaction of feeding different concentrations of cysteine and of lactate a Design of Experiments (DoE) study was conducted. A DoE study is a data collection and analysis tool that allows varying multiple input factors and determines their combined and single effects on different output parameters. Thus, this kind of study can identify interactions of multiple factors in a process by altering the levels of multiple inputs simultaneously in the process.

The DoE study was based on an I-optimal design and included the factors cystine (as a second feed with 17.2 g/L cystine (corresponding to ≈143 mM cysteine)) in a feeding-range from 0 to 1.67 ml/L/day (i.e., 0, 0.84 and 1.67 ml/L/day, corresponding to 0.12 and 0.24 mM/day) and sodium lactate (included in the regular feed with 30 ml/L/day) at a stock solution between 0 and 30 g/L (i.e. 0, 15 or 30 g/L, corresponding to 0, 0.133 and 0.267 M and a daily addition of 0, 4 and 8 mM/day). Since cysteine was added with the feed medium (6.3 mM at 30 ml/L/day; corresponding to a daily addition of 0.19 mM/day), the total daily addition of cysteine in the samples referred to as 0, 0.84 and 1.64 are 0.19, 0.31 and 0.43 mM/day, respectively.

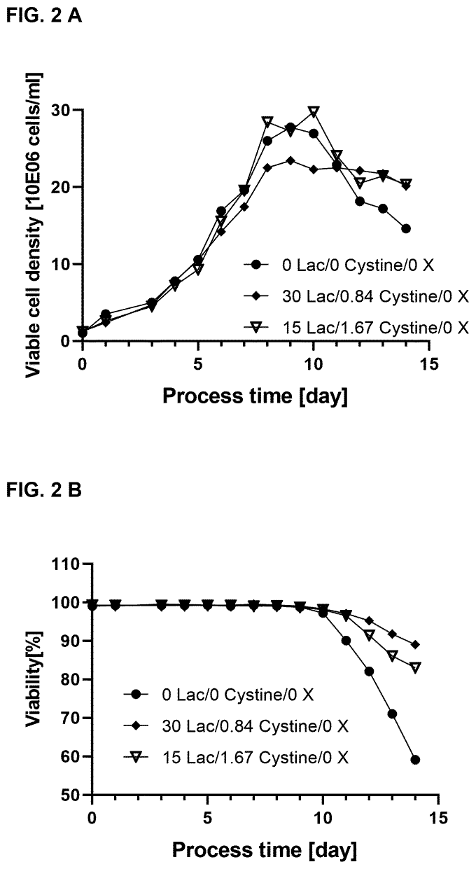

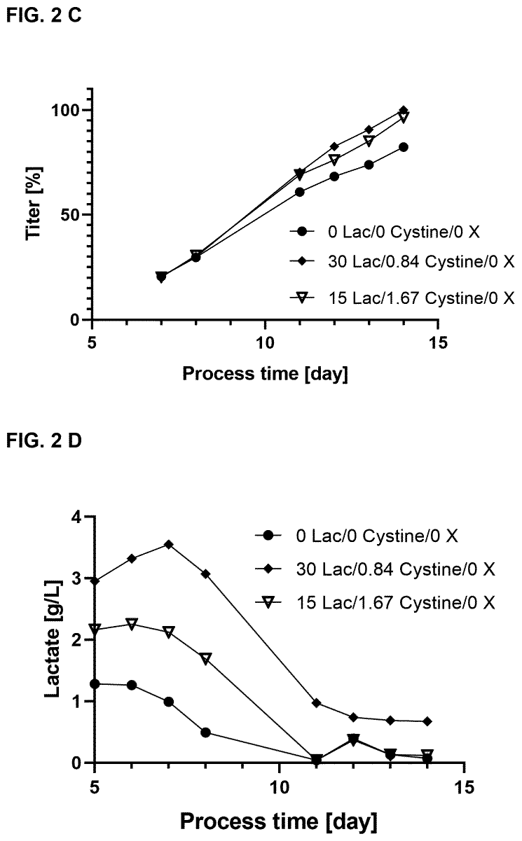

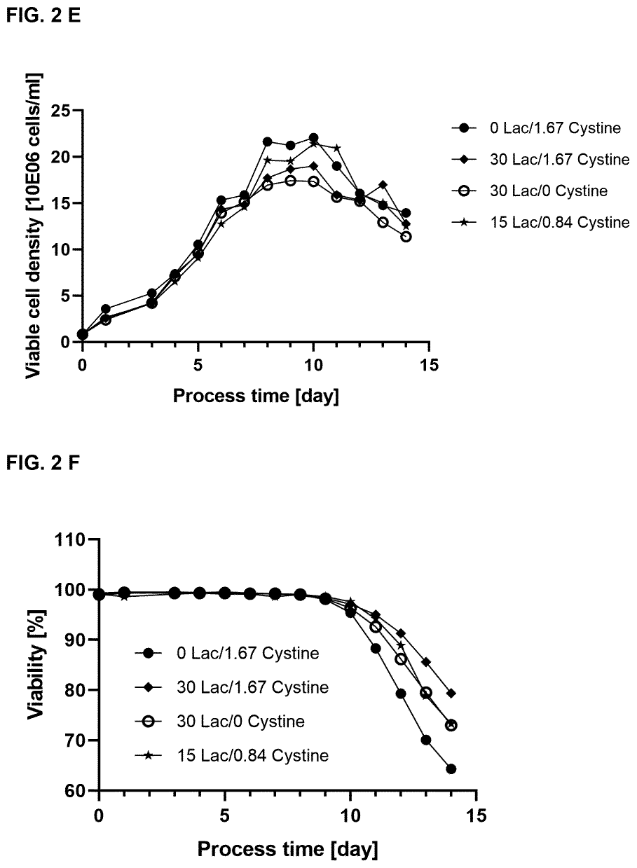

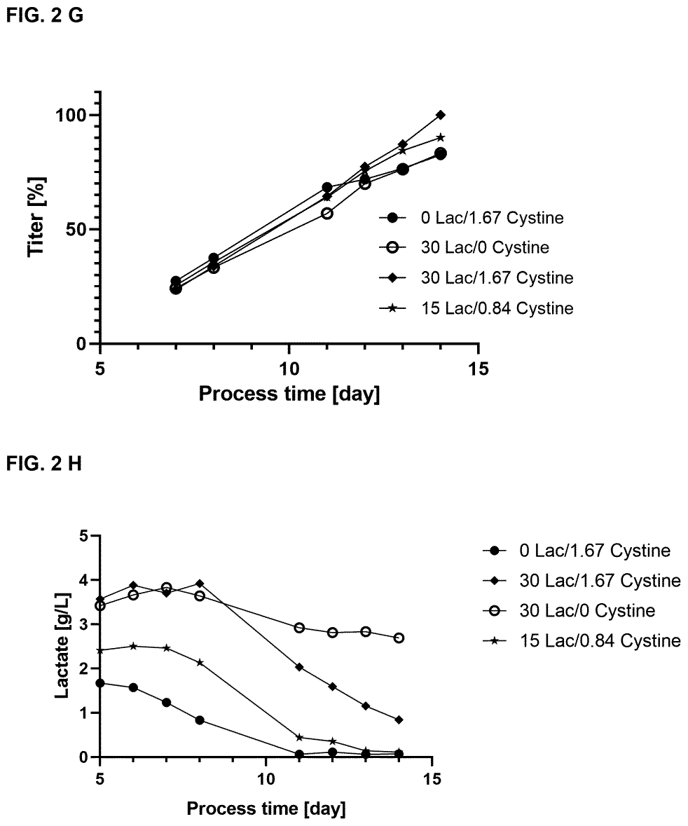

The effect of cysteine and/or lactate feeds on VCD, viability, product titer and lactate concentration in a regular process for cell lines A are shown in FIG. 2A-D and for cell line B in FIG. 2E-H. For both cell lines a beneficial effect on titer FIGS. 2C and G) as well a cell viability (Figures B and F) was observed with the lactate and cystine feed.

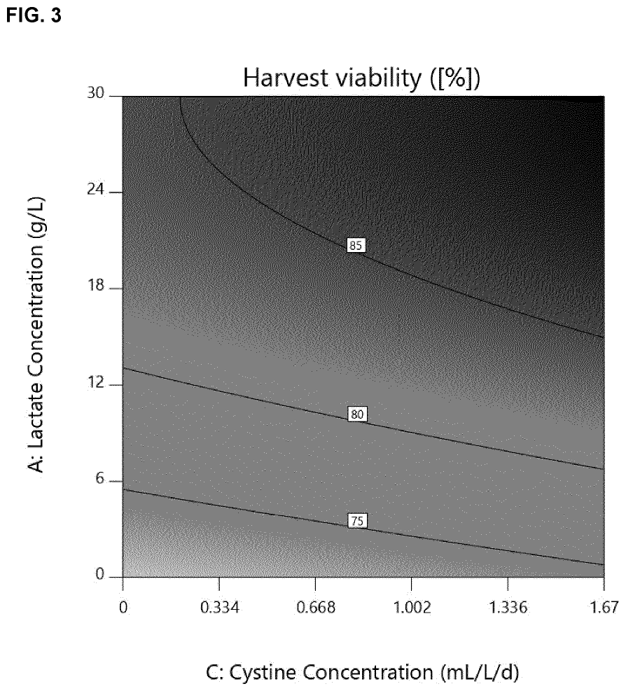

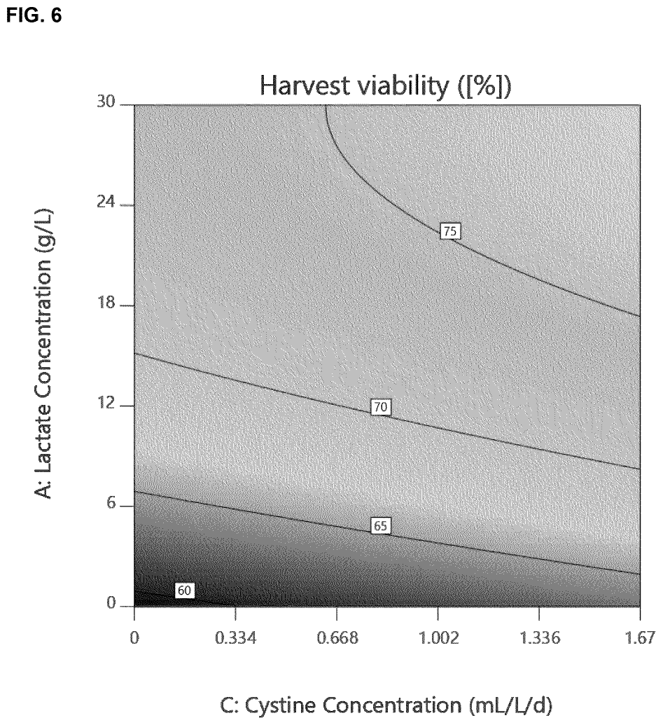

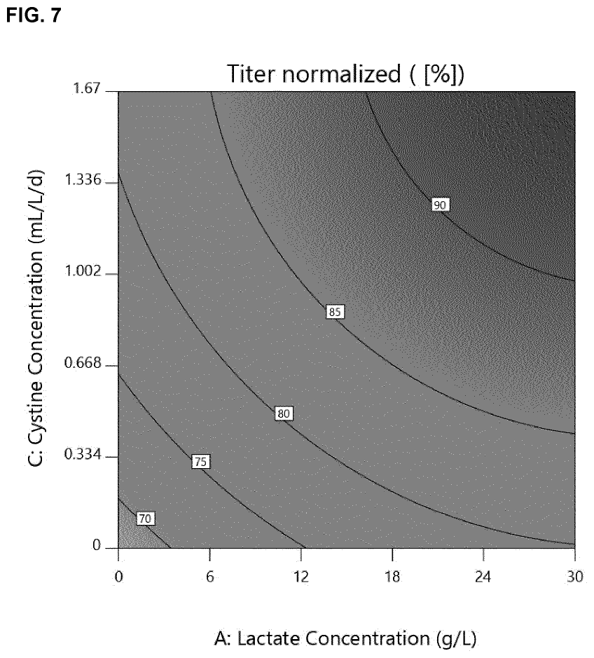

The positive effects of combinational lactate and cystine feeding on harvest viability and product titer at harvest are presented in DoE contour plots (cell line A: FIGS. 3 and 4; cell line B: FIGS. 6 and 7). Higher product titers were achieved using cell line B and the product titer in FIGS. 4 and 7 is provided as normalized [%] to the highest titer in the experiments, i.e., for cell line B. Using different concentrations for cell line B showed that highest product titers could be obtained at high lactate and high cysteine. Similar results were shown for cell line A, also demonstrating that high lactate and high cysteine increase harvest viability and product titer.

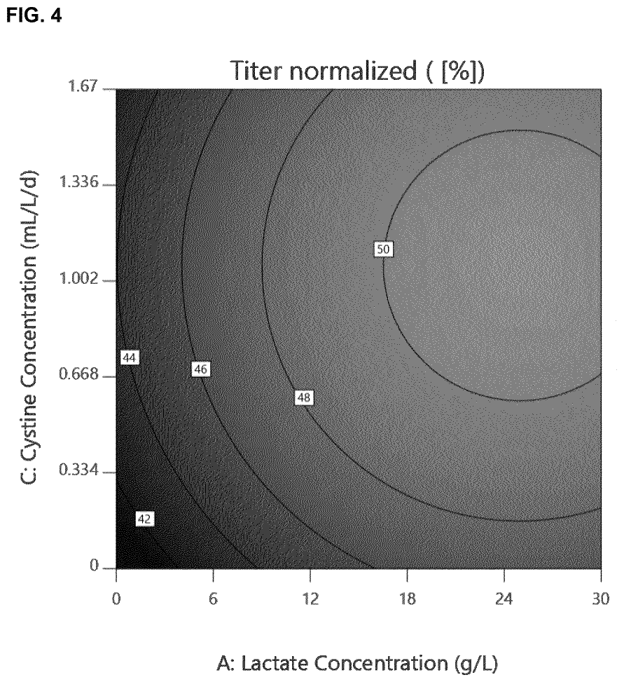

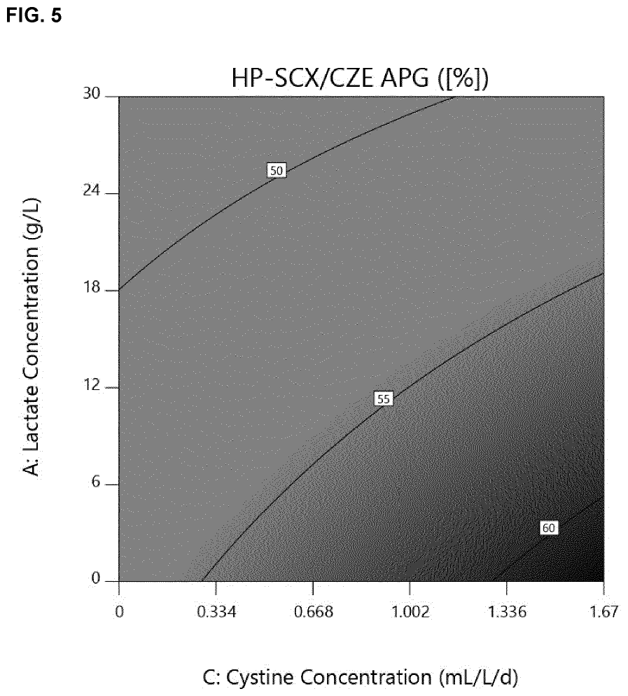

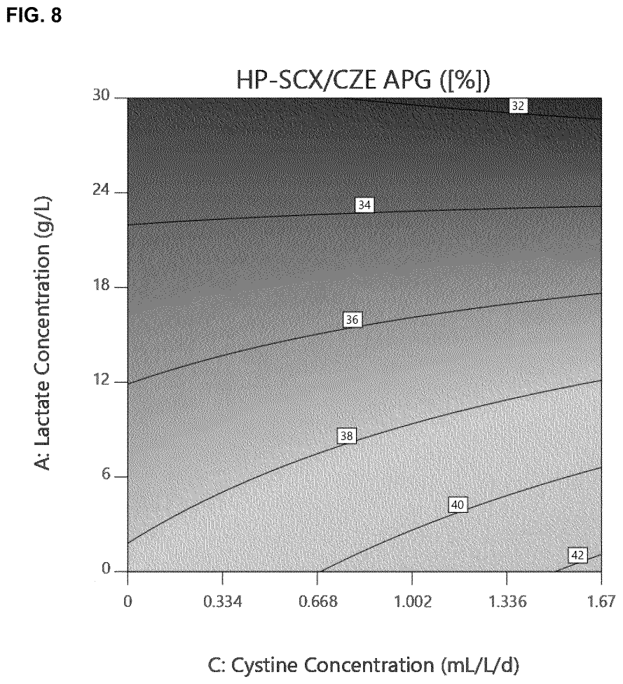

Cysteine as a known antioxidant is also known to increase acidic charge variants of antibodies. Therefore, the effects of combinational lactate and cysteine feeding on product quality attributes such as acidic charge variants (acidic peak group (APG)), low molecular weight species (LMWs) and high mannose species were determined. Surprisingly, positive effects of lactate feeding on APG, LMWs and high mannose structures were demonstrated as may be taken from FIGS. 5 and 8 (APGs) 9 (high mannose) and 10 (LMWs).

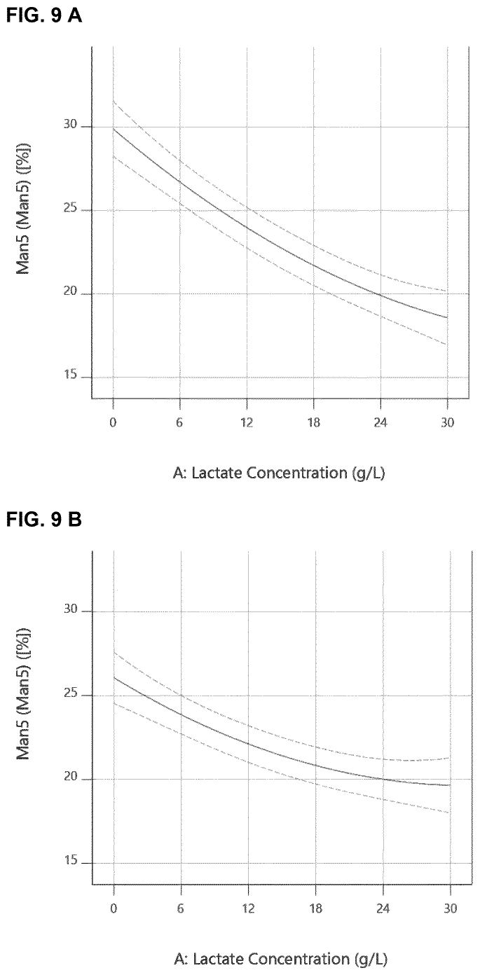

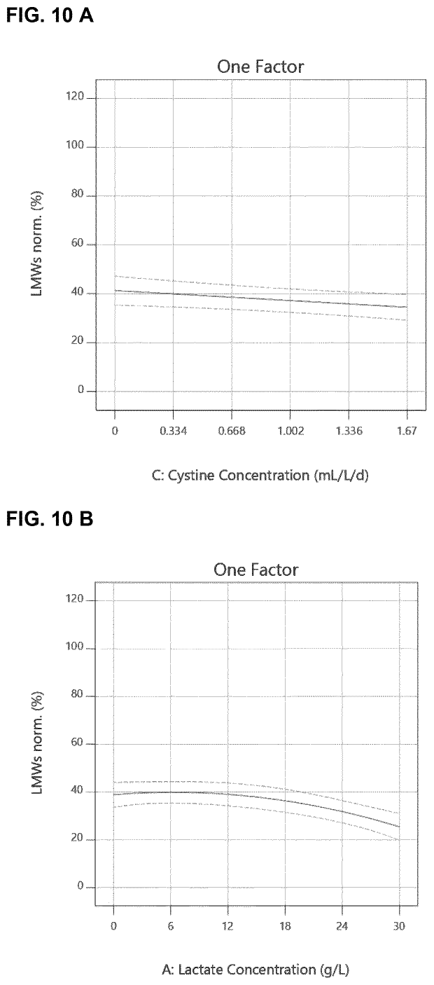

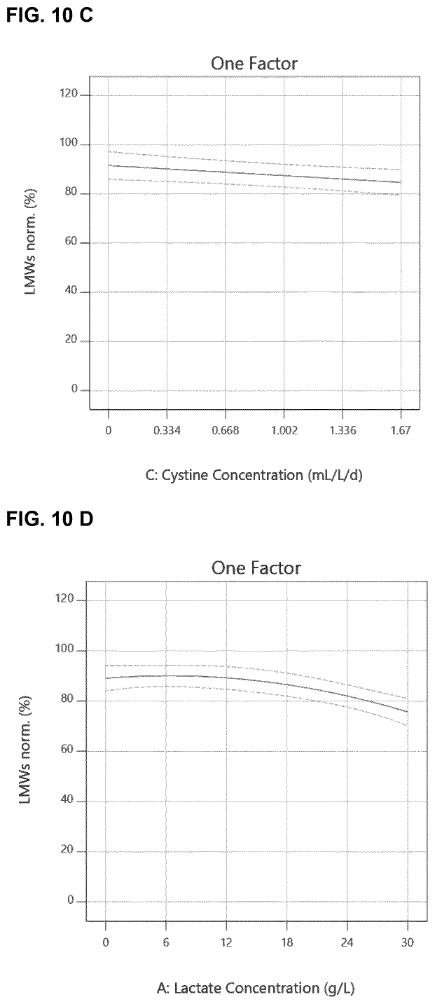

FIGS. 5 and 8 show the APGs for cell lines A and B, respectively as a function of lactate and cystine feeding. As can be seen from FIGS. 5 and 8 the increase in APGs due to cysteine feeding can be strongly reduced through additional lactate feeding. Further, as may be taken from FIGS. 9A and B, the mannose 5 structures (Mans) of antibodies can be reduced with increasing lactate concentrations for two different IgG1 antibodies produced by cell line A and cell line B. Finally, FIG. 10 shows the LMWs normalized to the highest value of the DoE (obtained with cell line B), for cell lines A and B as a function of cysteine (A and C) and lactate (B and D) concentrations. As can be seen from FIG. 10 the LMWs of the produced antibodies were reduced with increasing lactate or cystine feeding, resulting in a synergistic effect of reduced LMWs with both high lactate and high cysteine concentrations.

### Example 3: Reproducibility of Results with Additional Cell Lines

Four additional CHO cell lines including CHO-DG44 GS and CHO-K1 GS cells, producing a monoclonal antibody (mAb) were cultivated in an ambr15 bioreactor system. Cell lines C and F each produce an IgG4 monoclonal antibody with different specificity and cell lines D and E each produce an IgG1 monoclonal antibody with different specificity. The seed train cultures were processed in shake flasks and the seeding cell densities were set at 0.7×10E06 cells/ml. The fed-batch cultivations were conducted for 14 days. Feed medium was added continuously from day 2 until the end of the processes and glucose was added to the processes on demand as described in Example 2. In addition, the processes were performed with variable feeding using a feed medium comprising cysteine (1.1 g/L cysteine HCl H₂O; 0.0063 M) and lactate (267 mM) or a feed medium comprising cysteine, but no lactate (added as a regular continuous feed with 30 ml/L/day of the culture starting volume, Feed 1) and/or a second cysteine feed (Feed 2), as shown in Table 3. The lactate has been obtained as sodium lactate or as lactic acid, which has been titrated with NaOH to provide sodium lactate prior to addition to the feed medium. Cultivation samples were taken every 24 hours and cell counting and cell viability determination was performed using a Cedex HiRes analyzer (Roche, Germany). Glucose, lactate and antibody concentrations were determined using a Konelab Prime60i (Thermo Scientific, USA) or Biosen S-line (EKF-diagnostics GmbH, UK).

TABLE 3
Experimental set ups for reproducibility experiments

| | | Sodium lactate | Cystine |
|---|---|---|---|
| | | concentration | concentration |
| Experiment | Number of | in Feed 1 | in Feed 2 |
| ID | replicates | (30 ml/L/day) | (2 ml/L/day) |
| Control | 2 | 0 | — |
| w Cys | 2 | 0 | 14.37 g/L |
| w Lac | 2 | 30 g/L | — |
| w Cys/Lac | 2 | 30 g/L | 14.37 g/L |

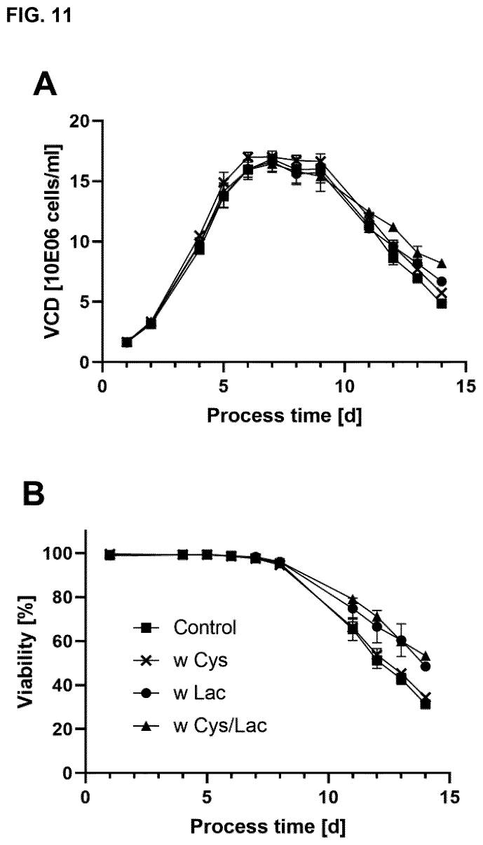

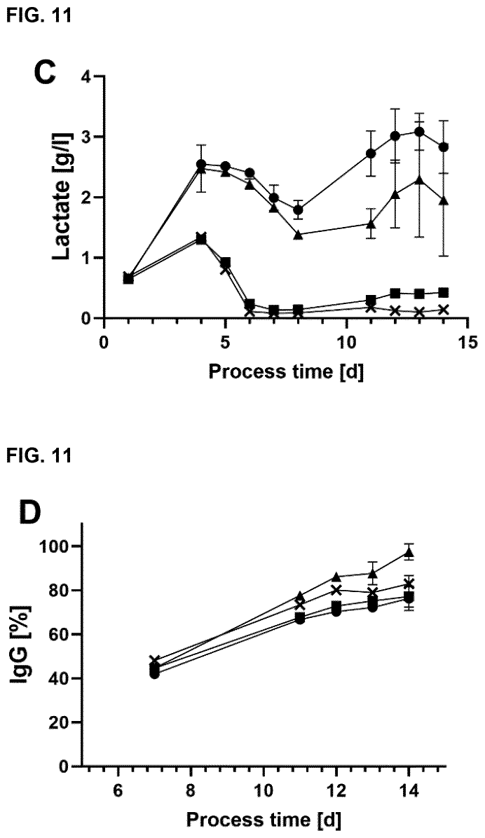

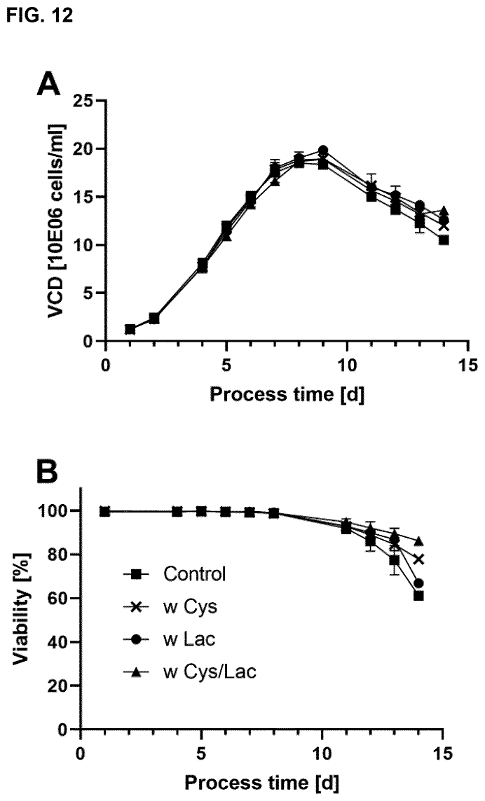

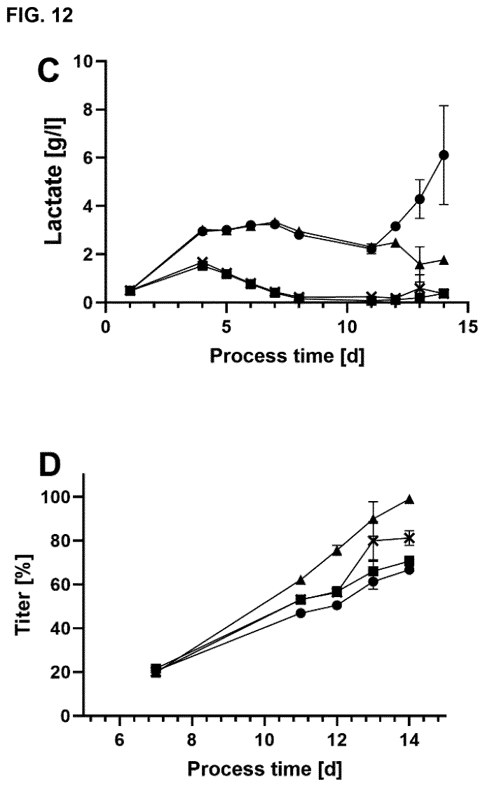

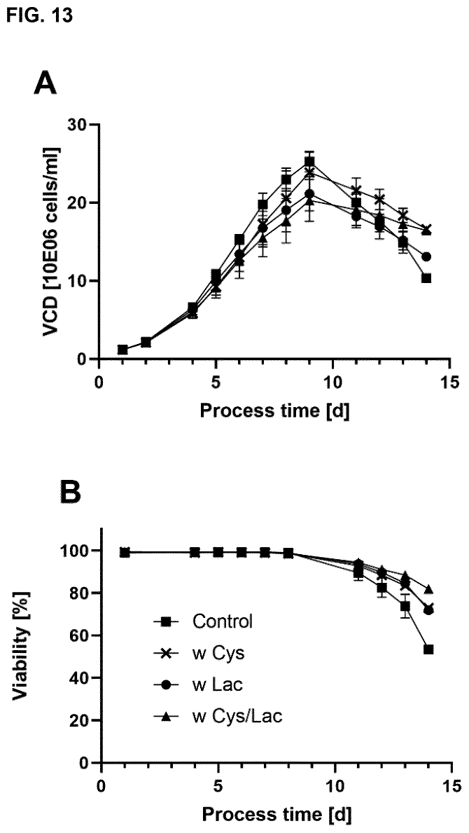

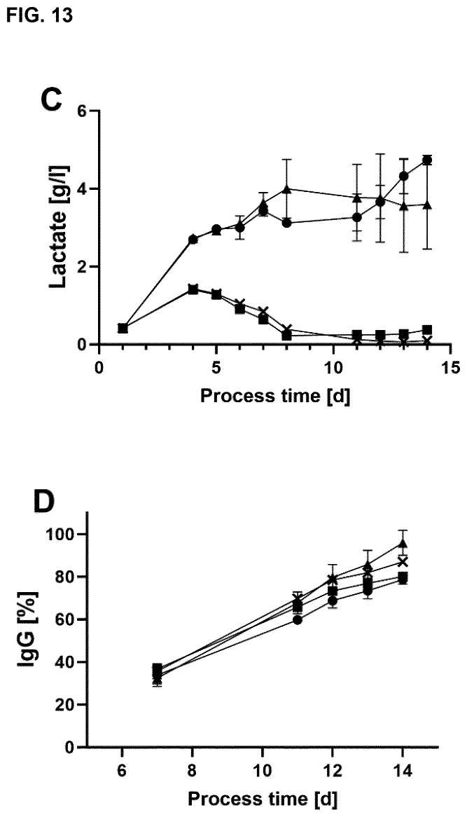

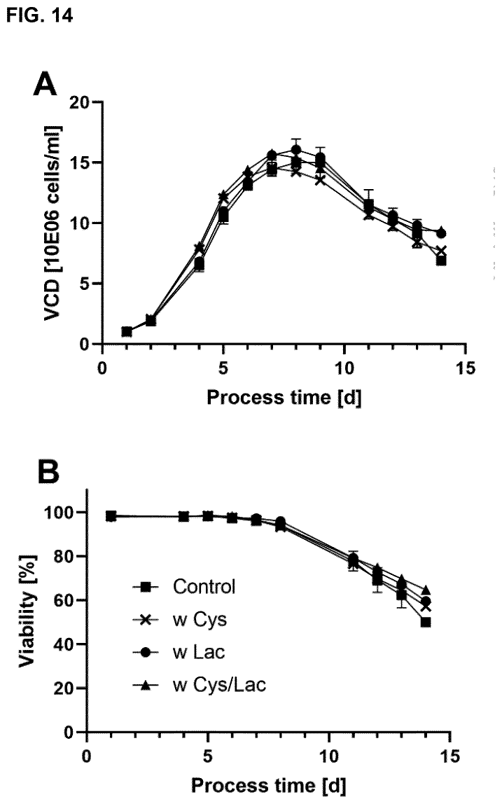

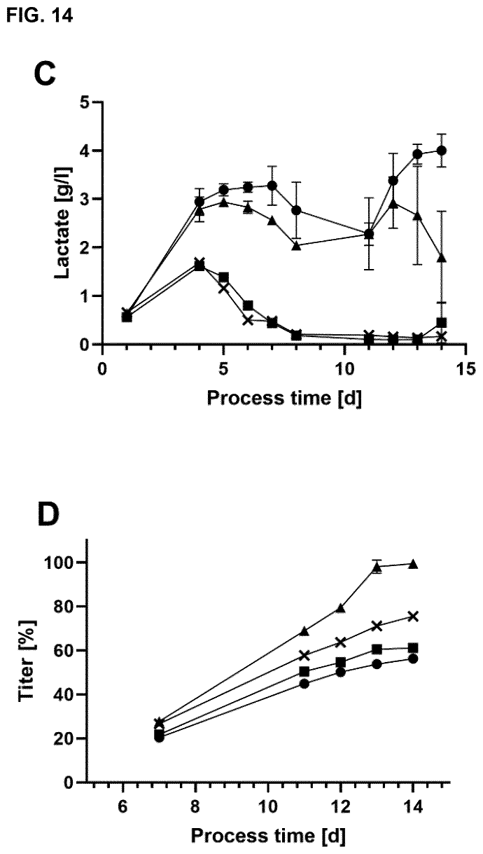

The experiments with four additional CHO cell lines confirmed the positive effect of the combination of lactate and cystine feeding on process performance. The highest cell viabilities and product titers were obtained with a combination of lactate and cystine feeding in all four cell lines (see FIG. 11 to FIG. 14).

### Example 4: DoE Optimization of uHSD Processes

The two CHO-K1 GS cell lines A and B, producing an IgG1 monoclonal antibody (mAb) were cultivated in an ambr 250 bioreactor system. Seed train cultures were processed in shake flasks until the N-1 stage which was processed in 2L single-use bioreactor systems in a perfusion mode. Fed-batch cultivations were conducted for 14 days. Feed media (comprising 1.1 g/L cysteine HCl H₂O (0.0063 M cysteine) and 0, 15 or 30 g/L sodium lactate (0, 0.133 or 0.267 M lactate) was added continuously from day 0 until the end of the processes. Seeding cell densities (SCD) were set between 5 to 10×10E06 cells/ml. Glucose was added to the proceses on demand. Cultivation samples were taken every 24 hours and cell counting and cell viability determination was performed using a Cedex HiRes analyzer (Roche, Germany). Glucose and lactate were determined using a Konelab Prime60i (Thermo Scientific, USA). Antibody concentration was determined with a Protein-A HPLC method (Thermo Scientific, USA).

The Design of Experiments (DoE) study was based on an I-optimal design including the factors cystine (as a second feed with 5.98 g/L cystine (corresponding to ˜49.83 mM) in a feeding-range from 0 to 4.8 ml/L/day (i.e., 0, 2.4 and 4.8 ml/L/day, corresponding to 0, 0.12 and 0.24 mM/day) and sodium lactate (included in the regular feed with 45 ml/L/day from day 0 to day 9 and with 25 ml/L/day from day 9 to day 14 of the culture starting volume) between 0 and 30 g/L (i.e., 0, 15 and 30 g/L; corresponding to 0, 0.133 and 0.267 M and a daily addition of 5.98 and 11.96 mM/day at 45 ml/L/day and of 3.32 and 6.64 mM/day at 25 ml/L/day). Since cysteine was also added with the feed medium (6.3 mM at 45 ml/L/day; corresponding to a daily addition of 0.28 mM/day and at 25 ml/L/day, corresponding to a daily addition of 0.16 mM/day), the total daily addition of cysteine in the samples adding 0, 2.4 and 4.8 ml/L/day in the second feed are 0.28, 0.4 and 0.52 mM/day at a daily addition of 45 ml/L/day and 0.16, 0.28 and 0.4 mM/day at a daily addition of 25 ml/L/day, respectively.

TABLE 4
Experimental set up

| Sample (cell A) | 1 | 2 | 3 | 4 | 5 | 6 | 7 | 8 | 9 | 10 | 11 | 12 |
|---|---|---|---|---|---|---|---|---|---|---|---|---|
| SCD | 5 | 5 | 5 | 7.5 | 7.5 | 7.5 | 7.5 | 10 | 10 | 10 | 10 | 10 |
| Sodium lactate g/L | 15 | 0 | 30 | 0 | 15 | 30 | 15 | 0 | 15 | 0 | 15 | 30 |
| Cystine 5.98 g/L | 2.4 | 4.8 | 2.4 | 0 | 2.4 | 2.4 | 4.8 | 0 | 0 | 2.4 | 2.4 | 4.8 |
| [ml/L/day] | | | | | | | | | | | | |

| Sample (cell B) | 1 | 2 | 3 | 4 | 5 | 6 | 7 | 8 | 9 | 10 |
|---|---|---|---|---|---|---|---|---|---|---|
| SCD | 5 | 5 | 5 | 5 | 7.5 | 7.5 | 7.5 | 10 | 10 | 10 |
| Sodium lactate g/L | 0 | 0 | 30 | 30 | 30 | 15 | 15 | 0 | 15 | 30 |
| Cystine 5.98 g/L | 0 | 2.4 | 0 | 4.8 | 0 | 2.4 | 4.8 | 0 | 4.8 | 2.4 |
| [ml/L/day] | | | | | | | | | | |

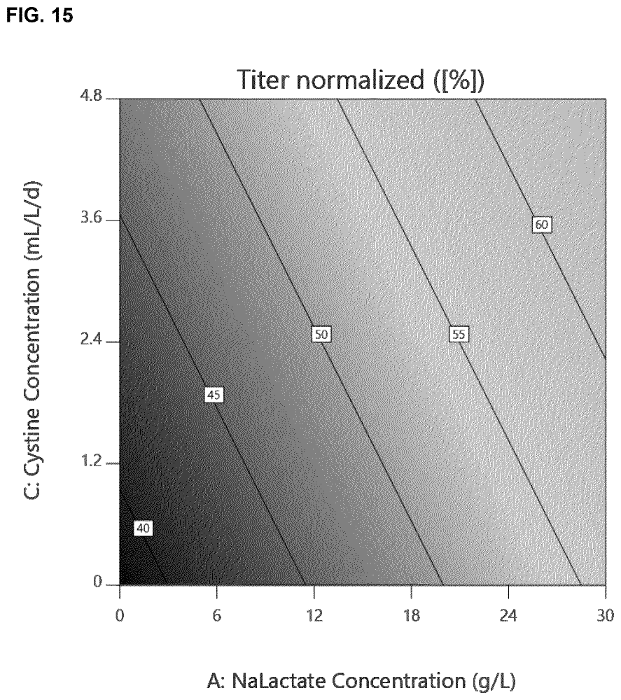

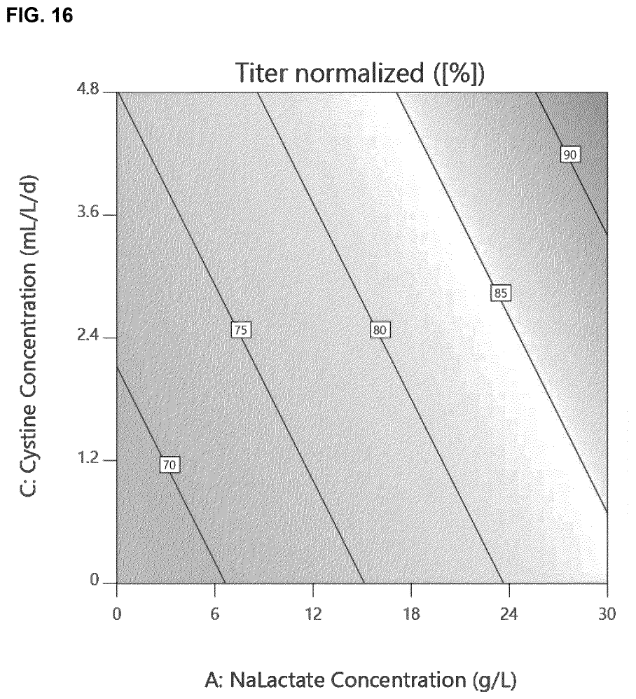

The positive effects of combinational lactate and cystine feeding on the product titer are presented in the DoE contour plots in FIG. 15 for cell line A and FIG. 16 for cell line B at harvest at day 14. Higher product titers were achieved using cell line B and the product titer in FIGS. 15 and 16 is provided as normalized [%] to the highest titer in the experiments, i.e., for cell line B. Highest product titers were obtained at high lactate and high cysteine feeding for both cell lines tested.

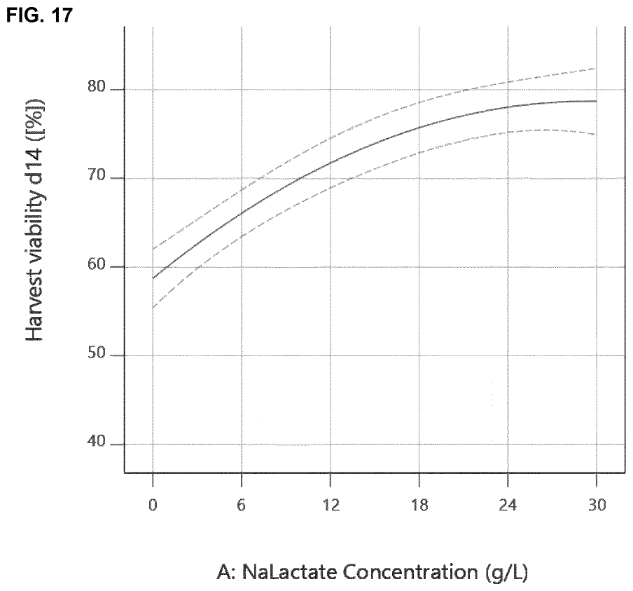

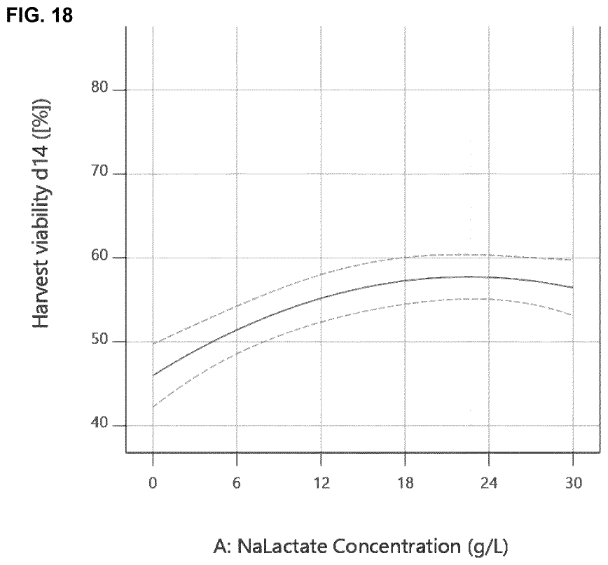

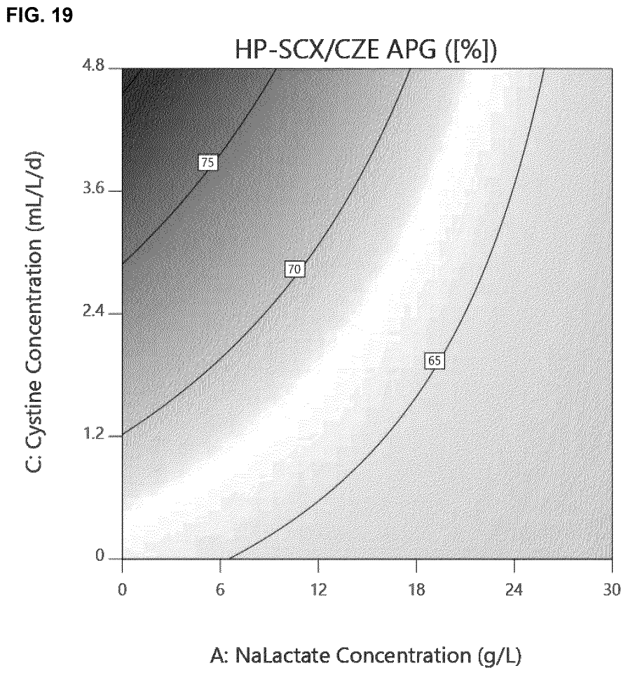

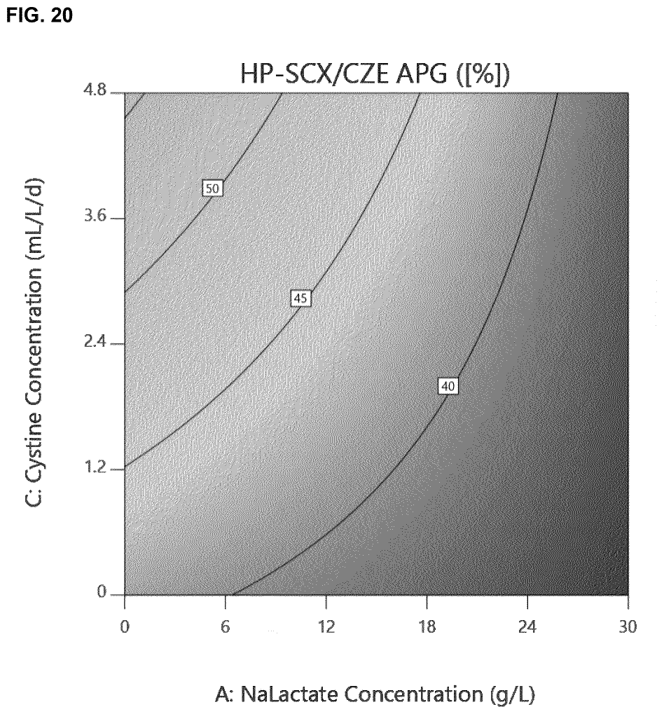

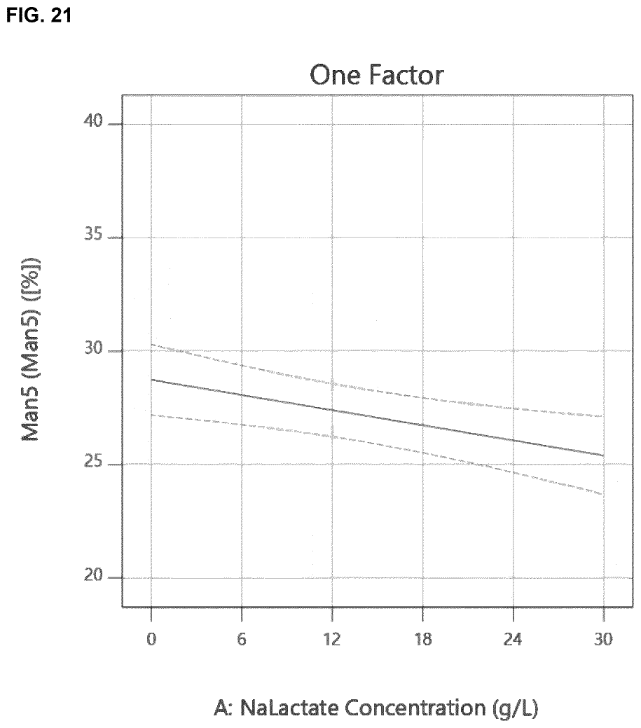

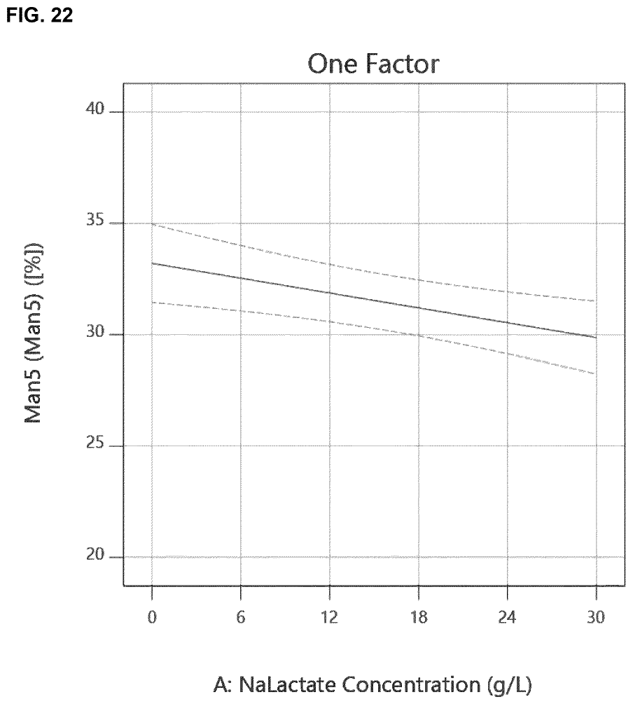

Posititve correlations of lactate feeding with harvest viability are further presented in FIGS. 17 and 18. The goodness of fit R2 and goodness of prediction Q2 are presented for each model. The effects on product quality attributes such as acidic charge variants and high mannose at harvest are presented in FIG. 19 to FIG. 22. It has been shown for both cell lines that an increase in acidic charge variants (APGs) due to high cysteine feeding, were strongly reduced by the additional lactate feeding.

1. A method of producing a product of interest in a fed-batch process comprising:
a) providing mammalian cells comprising a nucleic acid encoding a product of interest;
b) inoculating the mammalian cells in a basal medium to provide a cell culture;
c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher;
d) culturing the mammalian cells in the cell culture medium under conditions that allow expression of the product of interest; and
e) optionally isolating the product of interest.

2. The method according to claim 1, wherein the molar ratio of lactate/cysteine is about 10:1 to 50:1.

3. The method according to claim 1, wherein
a) the lactate is added at 3 mmol/L/day or higher;
b) the lactate in the cell culture medium is maintained at 0.5 g/L or higher or between 2 and 4 g/L;
c) the cysteine is provided as cysteine or a salt and/or hydrate thereof, cystine or a salt thereof or a dipeptide or tripeptide comprising cysteine; and/or
d) the cysteine is added at 0.25 mM/day or higher.

4. The method according to claim 1, wherein the product of interest is a heterologous protein or a recombinant virus.

5. The method according to claim 1, wherein the nucleic acid encodes a heterologous protein and
(a) the product titers and/or cell specific productivity is increased compared to the product titers and/or cell specific productivity of the heterologous protein produced by the same method, wherein the feed medium adds cysteine at or below 0.19 mM/day in the absence of lactate;
(b) the relative amount of high mannose structures in a population of the heterologous protein is reduced compared to a population of the heterologous protein produced by the same method, wherein the feed medium adds cysteine at or below 0.19 mM/day in the absence of lactate; and/or
(c) the relative amount (of total) of acidic species in a population of the heterologous protein is reduced compared to a population of the heterologous protein produced by the same method, wherein the feed medium adds the same concentration of cysteine in the absence of lactate.

6. The method of claim 1, wherein the basal medium and the feed medium is serum-free and chemically defined.

7. The method of claim 1, wherein the heterologous protein is an antibody or an antigen-binding fragment thereof, a bispecific antibody, a trispecific antibody or a fusion protein.

8. A method of culturing mammalian cells in a fed-batch process comprising:
a) providing mammalian cells comprising a nucleic acid encoding a product of interest;
b) inoculating the mammalian cells in a basal medium to provide a cell culture;
c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; and
d) culturing the mammalian cells in the cell culture medium under conditions that allow expression of the product of interest.

9. A method of reducing acidic species in a heterologous protein produced in a fed-batch process comprising:
a) providing mammalian cells comprising a nucleic acid encoding a heterologous protein;
b) inoculating the mammalian cells in a basal medium to provide a cell culture;
c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher;
d) culturing the mammalian cells in the cell culture medium under conditions that allow expression of the heterologous protein; and
e) optionally isolating the heterologous protein;
wherein the relative amount of acidic species in a population of the heterologous protein is reduced compared to a population of the heterologous protein produced by the same method wherein the feed medium adds the same concentration of cysteine in the absence of lactate.

10. A method of reducing high mannose structures in a heterologous protein produced in a fed-batch process comprising:
a) providing mammalian cells comprising a nucleic acid encoding a heterologous protein;
b) inoculating the mammalian cells in a basal medium to provide a cell culture;
c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher; and
d) culturing the mammalian cells in the cell culture medium under conditions that allow expression of the heterologous protein;
wherein the relative amount of the high mannose structures in a population of the heterologous protein is reduced compared to a population of the heterologous protein produced by the same method wherein the feed medium adds the cysteine at or below 0.19 mM/day in the absence of lactate.

11. A method of preventing negative effects of cysteine on product quality characteristics when producing a heterologous protein in a fed-batch process comprising:
a) providing mammalian cells comprising a nucleic acid encoding a heterologous protein;
b) inoculating the mammalian cells in a basal medium to provide a cell culture;
c) adding a feed medium comprising adding one or more feed supplements to the cell culture, wherein the feed medium adds lactate and cysteine at a molar ratio (mmol×L⁻¹×day⁻¹/mmol×L⁻¹×day⁻¹) of lactate/cysteine of about 8:1 to about 50:1 to the basal medium resulting in a cell culture medium or to the resulting cell culture medium, wherein the cysteine is added at 0.225 mM/day or higher;
d) culturing the mammalian cells in the cell culture medium under conditions that allow expression of the heterologous protein; and
e) optionally isolating the heterologous protein from the mammalian cells;
wherein the negative effects on product quality characteristics in a population of the heterologous protein are reduced compared to a population of the heterologous protein produced by the same method wherein the feed medium adds the same concentration of cysteine in the absence of lactate.

12. A heterologous protein produced by the method of claim 9.

13-15. (canceled)

16. The method of claim 1, wherein the fed-batch process comprises culturing a mammalian cell, wherein the mammalian cell is a HEK293 cell or a CHO cell or a CHO derived cell.

17. A feed medium for mammalian cell fed-batch culture comprising lactate and cysteine at a molar ratio (mM/mM) of lactate/cysteine of about 8:1 to about 50:1.

18. (canceled)

19. A heterologous protein produced by the method of claim 10.

20. A heterologous protein produced by the method of claim 11.
